(19) United States
(12) Patent Application Publication
(10) Pub. No.: US 2010/0183674 A1
(43) Pub. Date: Jul. 22, 2010

(54) COMPOSITIONS COMPRISING YERSINIA PESTIS ANTIGENS

(76) Inventors: Renata Maria Grifantini, Siena (IT); Guido Grandi, Milano (IT); Maria Scarselli, Siena (IT); Erika Bartolini, Castelnuovo Berardenga (IT); Elisabetta Frigimelica, Mogliano Veneto (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/733,488

(22) PCT Filed: Sep. 4, 2008
(86) PCT No.: PCT/IB2008/003081

§ 371 Date: Mar. 4, 2010

(30) Foreign Application Priority Data
Sep. 4, 2007 (GB) 0717187.9

(51) Int. Cl. A61K 39/02; C07K 16/12; A61P 31/04; A61P 37/04

(52) U.S. Cl. 424/234.1; 530/387.1

(57) ABSTRACT

Disclosed are several Y. pestis antigens that are particularly suitable for immunisation purposes, particularly when used in combinations.

### GOVERNMENT SUPPORT

The invention was supported, in whole or in part, by Grant No. 1U01 A156513-01 from the US National Institute of Allergy and Infectious Diseases. The US Government may have certain rights in the invention.

This application claims priority from United Kingdom patent application 0717187.9, filed 4 Sep. 2007, the entire contents of which are incorporated herein by reference.

### TECHNICAL FIELD

This invention is in the fields of immunology and vaccinology. In particular, it relates to antigens derived from Yersinia pestis and their use in immunisation.

### BACKGROUND ART

There are three recognised forms of plague in man: bubonic, septicaemic and pneumonic. All are caused by the Yersinia pestis bacterium, which has also been known as Pasteurella pestis, Bacterium pestis and Pestisella pestis. Y. pestis is endemic on every continent in the world except Australia [1], and results in around 1700 cases of plague a year. It is a Gram-negative non-motile aerobic bacillus.

Bubonic plague is the most common form of disease and arises following a bite from a flea which has fed previously on an infected animal. From the initial site of infection the bacteria are disseminated to the draining lymph nodes, which become swollen and tender to form buboes.

Septicaemic plague occurs when there is bacteremia without the development of buboes and is characterised by an elevated temperature, chills, headache, malaise and gastrointestinal disturbances. Because of the generalised nature of these symptoms a diagnosis of plague is often delayed, and even with medical intervention 50% of patients die, probably as a result of the induction of the systemic inflammatory response syndrome.

The most feared form of plague arises when there is colonisation of the alveolar spaces leading to a pneumonia, causing the pneumonic plague. Pneumonic plague is transmitted by airborne droplets containing bacteria, generated by coughing, which can be inhaled by susceptible individuals. The pneumonic form of the disease is feared because of the rapidity with which the disease develops (1-3 days), the high mortality rate in infected individuals (about 100%) and the rapid spread of disease from man to man.

Due to the high infectivity and mortality of pneumonic plague, Y. pestis is considered to be a likely biological threat agent [2].

The only plague vaccine licensed in the United States is the ‘USP vaccine’, a preparation of formaldehyde-killed Y. pestis, but it is no longer produced. This vaccine relies on the F1 capsular protein as the main immunogen. While it has been shown to be effective against subcutaneous challenge, it is not effective against aerosol challenge [3], and unpleasant side effects have been reported. The vaccine also fails to protect against the F1⁻ variants of Y. pestis, which are equally virulent in rodents [4, 5] and which have been isolated from at least one fatal human case [6].

More recent studies have focused on recombinant subunit vaccines. Purified or recombinant F1 antigen may confer protection against both bubonic and pneumonic plague [7], as may the V antigen [8].

These studies indicate that development of an efficacious subunit vaccine based on recombinant Y. pestis proteins for use in man is feasible.

While the F1 and V antigens are promising candidates for inclusion in a prophylactic vaccine, it is unclear if these antigens alone will afford sufficient protection in humans, or whether they would be useful in immunotherapeutic vaccines. Thus reference 9 reports the identification of several further antigens for vaccine use.

Thus there remains a need to develop a broadly-protective multivalent vaccine against all potential variant and engineered strains [2].

### DISCLOSURE OF THE INVENTION

The inventors believe that an effective Y. pestis vaccine will require several antigenic components, and that these components may or may not include the F1 or V antigens.

With this in mind, they identified in reference 9 various surface-exposed Y. pestis antigens that are particularly suitable for immunisation purposes, particularly when used in combinations. The antigens are exposed on the bacterial surface and have been identified using “surface shaving” techniques or by detecting proteins that were labelled in situ on the cell surface Y. pestis proteins.

Thus the invention provides a composition comprising a combination of Y. pestis antigens, said combination comprising two or more (i.e. 2, or all 3) Y. pestis antigens selected from the group consisting of: (1) a YPO0512 antigen; (2) a YPO0563 antigen; and (3) a YPO3489 antigen. These three antigens form the “first antigen group”. Within the first antigen group, a YPO3489 antigen is preferred.

The invention also provides a composition comprising a combination of Y. pestis antigens, said combination comprising two or more (i.e. 2, 3, 4 or all 5) Y. pestis antigens selected from the group consisting of (1) a YPO0512 antigen; (2) a YPO0563 antigen; (3) a YPO3489 antigen; (4) a YPO4003 antigen; and (5) a YPO1604 antigen. These five antigens form the “second antigen group”, which includes the three antigens of the first antigen group. Within the second antigen group, a YPO3489 antigen, a YPO4003 antigen and/or a YPO1604 antigen are preferred.

The invention also provides a composition comprising a combination of Y. pestis antigens, said combination comprising one or more (i.e. 1, 2, 3, 4, 5, 6, 7, 8, 9, 10, 11, 12, 13, 14, 15, 16, 17, 18, 19, 20, or all 21) Y. pestis antigens from the group consisting of (1) a YPO0512 antigen; (2) a YPO0563 antigen; (3) a YPO3489 antigen; (4) a YPO4003 antigen; (5) a YPO1604 antigen; (6) a YPO3061 antigen; (7) a YPO3559 antigen; (8) a YPO3382 antigen; (9) a YPO0860 antigen; (10) a YPO0086 antigen; (11) a YPO3631 antigen; (12) a YPO2881 antigen; (13) a YPO3343 antigen; (14) a YPO3361 antigen; (15) a YPO3430 antigen; (16) a YPO1411 antigen; (17) a YPO3935 antigen; (18) a YPO0809 antigen; (19) a YPO1123 antigen; (20) a YPO3065 antigen; and (21) a YPO1070 antigen. These 21 antigens form the “third antigen group”, which includes the five antigens from the second antigen group. Within the third antigen group, a YPO3489 antigen, a YPO4003 antigen, a YPO1604 antigen, a YPO2881 antigen, a YPO0809 antigen, a YPO1123 antigen, a YPO1411 antigen, a YPO3935 antigen and/or a YPO1070 antigen are preferred.

The invention also provides a composition comprising a combination of Y. pestis antigens, said combination comprising one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36) Y. pestis antigens from the group consisting of (1) a YPO0102 antigen; (2) a YPO0570 antigen; (3) a YPO1053 antigen; (4) a YPO1435 antigen; (5) a YPO2674 antigen; (6) a YPO2292 antigen; (7) a YPO3050 antigen; (8) a YPO2615 antigen; (9) a YPO1507 antigen; (10) a YPO4111 antigen; (11) a YPO0015 antigen; (12) a YPO0195 antigen; (13) a YPO2342 antigen; (14) a YPO0501 antigen; (15) a YPO0502 antigen; (16) a YPO0819 antigen; (17) a YPO3644 antigen; (18) a YPO1746 antigen; (19) a YPO0351 antigen; (20) a YPO0468 antigen; (21) a YPO0203 antigen; (22) a YPO0216 antigen; (23) a YPO3536 antigen; (24) a YPO0233 antigen; (25) a YPO0067 antigen; (26) a YPO3643 antigen; (27) a YPO3375 antigen; (28) a YPO0494 antigen; (29) a YPO1052 antigen; (30) a YPO1906 antigen; (31) a YPO0663 antigen; (32) a YPO1222 antigen; (33) a YPO2905 antigen; (34) a YPO4070 antigen; (35) a YPPCP1.07 antigen; and (36) a YPMT1.42 antigen. These 36 antigens form the “fourth antigen group”, which does not overlap with the first, second or third antigen groups. Within the fourth antigen group, a YPO0502 antigen is preferred.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, or all 21) Y. pestis antigens selected from the third antigen group (preferably comprising an antigen from the second group, and more preferably from the first antigen group) and one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36) Y. pestis antigens of the fourth antigen group.

The immunogenicity of other Y. pestis antigens of known and unknown biological function may be improved by combination with one or more Y. pestis antigens from either the first antigen group and/or the second and/or the third antigen group and/or the fourth antigen group. Such other Y. pestis antigens of known and unknown biological function include a F1 antigen and/or a V antigen. These two antigens form the “fifth antigen group”.

Thus the invention provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, or all 21) Y. pestis antigens selected from the third antigen group (preferably comprising an antigen from the second group, and more preferably from the first antigen group) and one or two Y. pestis antigens from the fifth antigen group.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36) Y. pestis antigens selected from the fourth antigen group and one or two Y. pestis antigens from the fifth antigen group.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, or all 21) Y. pestis antigens selected from the third antigen group (preferably comprising an antigen from the second group, and more preferably from the first antigen group), one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36) Y. pestis antigens selected from the fourth antigen group, and one or two Y. pestis antigens from the fifth antigen group.

The invention also provides a composition comprising a combination of Y. pestis antigens, said combination comprising one or more (i.e. 1, 2, 3, 4, 5, 6, 7, 8, 9, 10, 11, 12, 13, 14, 15, 16, 17, 18, 19, 20, 21, 22, 23, 24, 25 or all 26) Y. pestis antigens from the group consisting of: (1) a YPO0457 antigen; (2) a YPO0514 antigen; (3) a YPO0694 antigen; (4) a YPO0805 antigen; (5) a YPO0982 antigen; (6) a YPO1354 antigen; (7) a YPO1408 antigen; (8) a YPO1792 antigen; (9) a YPO2506 antigen; (10) a YPO2713 antigen; (11) a YPO2950 antigen; (12) a YPO3026 antigen; (13) a YPO3417 antigen; (14) a YPO3551 antigen; (15) a YPO3646 antigen; (16) a YPO3982 antigen; (17) a YPO0065 antigen; (18) a YPO0499 antigen; (19) a YPO0505 antigen; (20) a YPO0500 antigen; (21) a YPO0503 antigen; (22) a YPO0506 antigen; (23) a YPO0508 antigen; (24) a YPO0509 antigen; (25) a YPO3579 antigen and (26) a YPO4040 antigen. These 26 antigens form the “sixth antigen group”, which does not overlap with the first, second, third, fourth or fifth antigen groups. Within the sixth antigen group, a YPO3982 antigen, a YPO0499 antigen and/or a YPO0505 antigen are preferred.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, 21) Y. pestis antigens selected from the third antigen group (preferably comprising an antigen from the second group, and more preferably from the first antigen group) and one or more (i.e. 1, 2, 3, 4, 5, 6, 7, 8, 9, 10, 11, 12, 13, 14, 15, 16, 17, 18, 19, 20, 21, 22, 23, 24, 25 or all 26) Y. pestis antigens of the sixth antigen group.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36) Y. pestis antigens of the fourth antigen group and one or more (i.e. 1, 2, 3, 4, 5, 6, 7, 8, 9, 10, 11, 12, 13, 14, 15, 16, 17, 18, 19, 20, 21, 22, 23, 24, 25 or all 26) Y. pestis antigens of the sixth antigen group.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, 21, 22, 23, 24, 25 or all 26) Y. pestis antigens selected from the sixth antigen group and one or two Y. pestis antigens from the fifth antigen group.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, or all 21) Y. pestis antigens selected from the third antigen group (preferably comprising an antigen from the second group, and more preferably from the first antigen group), one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36) Y. pestis antigens selected from the fourth antigen group, one or two Y. pestis antigens from the fifth antigen group, and one or more (i.e. 1, 2, 3, 4, 5, 6, 7, 8, 9, 10, 11, 12, 13, 14, 15, 16, 17, 18, 19, 20, 21, 22, 23, 24, 25 or all 26) Y. pestis antigens selected from the sixth antigen group.

The invention also provides a composition comprising a combination of Y. pestis antigens, said combination comprising one or more (i.e. 1, 2, 3, 4 or all 5) Y. pestis antigens from the group consisting of: (1) a YPO0496 antigen; (2) a YPO1224 antigen; (3) a YPO3553 antigen; (4) a YPO3987 antigen; and (5) a YPO2190 antigen. These 5 antigens form the “seventh antigen group”, which does not overlap with the first, second, third, fourth, fifth or sixth antigen groups.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, or all 21) Y. pestis antigens selected from the third antigen group (preferably comprising an antigen from the second group, and more preferably from the first antigen group) and one or more (i.e. 1, 2, 3, 4 or all 5) Y. pestis antigens of the seventh antigen group.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36) Y. pestis antigens selected from the fourth antigen group and one or more (i.e. 1, 2, 3, 4 or all 5) Y. pestis antigens of the seventh antigen group.

The invention also provides a composition comprising a combination of Y. pestis antigens, said combination including one or two Y. pestis antigens selected from the fifth antigen group and one or more (i.e. 1, 2, 3, 4 or all 5) Y. pestis antigens of the seventh antigen group.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, 21, 22, 23, 24, 25 or all 26) Y. pestis antigens selected from the sixth antigen group and one or more (i.e. 1, 2, 3, 4 or all 5) Y. pestis antigens of the seventh antigen group.

The invention also provides a composition comprising a combination of Y. pestis antigens, said combination including one or more (i.e. 1, 2, 3, 4, 5, 6, 7, 8, 9, 10, 11, 12, 13, 14, 15, 16, 17, 18, 19, 20, or all 21) Y. pestis antigens selected from the third antigen group (preferably comprising an antigen from the second group, and more preferably from the first antigen group), one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36) Y. pestis antigens selected from the fourth antigen group, one or two Y. pestis antigens from the fifth antigen group, one or more (i.e. 1, 2, 3, 4, 5, 6, 7, 8, 9, 10, 11, 12, 13, 14, 15, 16, 17, 18, 19, 20, 21, 22, 23, 24, 25 or all 26) Y. pestis antigens selected from the sixth antigen group and one or more (i.e. 1, 2, 3, 4 or all 5) Y. pestis antigens of the seventh antigen group. The invention also provides a composition comprising a combination of Y. pestis antigens, said combination comprising a antigens from the third antigen group, b antigens from the fourth antigen group, and c antigens from the fifth antigen group, wherein: a is selected from 0, 1, 2, 3, 4, 5, 6, 7, 8, 9, 10, 11, 12, 13, 14, 15, 16, 17, 18, 19, 20 or 21; b is selected from 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36; and c is selected from 0, 1 or 2; provided that a+b+c is at least 2 (e.g. 2, 3, 4, 5, 6, 7, 8, 9, 10, or more). Preferably a is not 0. Preferably c is not 0.

Such a composition may optionally comprise d antigens from the sixth antigen group, wherein d is selected from 0, 1, 2, 3, 4, 5, 6, 7, 8, 9, 10, 11, 12, 13, 14, 15, 16, 17, 18, 19, 20, 21, 22, 23, 24, 25 or 26; provided that a+b+c+d is at least 2 (e.g. 2, 3, 4, 5, 6, 7, 8, 9, 10, or more). Preferably a is not 0. Preferably c is not 0.

Such compositions may optionally comprise e antigens from the seventh antigen group, wherein e is selected from 0, 1, 2, 3, 4 or 5; provided that a+b+c+d+e is at least 2 (e.g. 2, 3, 4, 5, 6, 7, 8, 9, 10, or more). Preferably a is not 0. Preferably c is not 0.

The above compositions may also include further Y. pestis antigens that are not members of any of the first, second, third, fourth, fifth or sixth antigen groups. For example, the compositions may include a pesticin (YPPCP1.05c), a W antigen, a pH 6 antigen (YPO1303), a Fe or Mn superoxide dismutase (Fe YPO2386; Mn YPO4061), a YOP antigen (e.g. YPCD1.34c), an iron regulated membrane protein (e.g. YPO1313), a murine toxin (YPMT1.74), a hemin storage protein (e.g. YPO0281), etc. Preferably, a composition according to the invention may further comprise an OppA antigen (YPO2182) as described in reference 10.

There is an upper limit to the number of Y. pestis antigens which will be found in compositions of the invention. Preferably, the number of Y. pestis antigens in a composition of the invention is less than 20 (e.g. less than 19, less than 18, less than 17, less than 16, less than 15, less than 14, less than 13, less than 12, less than 11, less than 10, less than 9, less than 8, less than 7, less than 6, less than 5, less than 4, or less than 3). In particular, the number of Y. pestis antigens in a composition of the invention is preferably less than 6, less than 5, or less than 4.

Preferred antigens selected from the third antigen group are those in the second antigen group, and preferred antigens selected from the second antigen group are those in the first antigen group.

Preferred compositions according to the invention comprise two or more (i.e. 2, 3, 4, 5, 6, 7, 8, 9, 10, 11, 12 or all 13) of a YPO0499 antigen, a YPO0502 antigen, a YPO0505 antigen, a YPO0809 antigen, a YPO1070 antigen, a YPO1123 antigen, a YPO1604 antigen, a YPO2881 antigen, a YPO3489 antigen, a YPO1411 antigen, a YPO3935 antigen, a YPO3982 antigen and a YPO4003 antigen.

Preferred compositions according to the invention may comprise one or more (i.e. 1, 2, 3 or all 4) of a YPO0499 antigen, a YPO1604 antigen, a YPO3489 antigen and a YPO4003 antigen. Preferably, a composition according to the invention comprises all four of a YPO0499 antigen, a YPO1604 antigen, a YPO3489 antigen and a YPO4003 antigen.

Further preferred compositions according to the invention may comprise one or more (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or all 31) of a YPO0065 antigen, a YPO0086 antigen, a YPO0496 antigen, a YPO0499 antigen, a YPO0501 antigen, a YPO0502 antigen, a YPO0505 antigen, a YPO0809 antigen, a YPO0860 antigen, a YPO1070 antigen, a YPO1123 antigen, a YPO1224 antigen, a YPO1411 antigen, a YPO1604 antigen, a YPO2506 antigen, a YPO2881 antigen, a YPO3935 antigen, a YPO3061 antigen, a YPO3065 antigen, a YPO3382 antigen, a YPO3489 antigen, a YPO3551 antigen, a YPO3553 antigen, a YPO3579 antigen, a YPO3631 antigen, a YPO3982 antigen, a YPO4003 antigen, a YPO3987 antigen, a YPO1354 antigen, a YPO2190 antigen and a YPO3417 antigen. Particularly preferred compositions according to the invention comprise (i) a YPO0499 antigen, a YPO0502 antigen and a YPO0505 antigen, (ii) a YPO1070 antigen, a YPO1123 antigen, a YPO2881 antigen and a YPO0809 antigen, or (iii) a YPO1411 antigen, a YPO3935 antigen and a YPO3982 antigen.

Further preferred compositions according to the invention may comprise one or more of a YPO0468 antigen (DnaK), a YPO0351 antigen (GroEL), a YPO0203 antigen (EF-Tu) and a YPO1222 antigen (OmpC). Compositions may also optionally comprise a YPO1792 antigen (FlhE).

Such preferred compositions may also optionally comprise one or both of the Y. pestis F1 and V antigens.

### First Antigen Group

### (1) YPO0512

The ‘YPO0512’ sequence was annotated in reference 11 as ‘putative lipoprotein’ (see GI:16120843). For reference purposes, the amino acid sequence of full-length YPO0512 as found in the Y. pestis CO92 strain is given as SEQ ID NO:1 herein. Furthermore, it is postulated that YPO0512 forms part of a Type Three Secretion System (TTSS).

Preferred YPO0512 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:1; and/or (b) that is a fragment of at least n consecutive amino acids of SEQ ID NO:1, wherein n is 7 or more (e.g. 8, 10, 12, 14, 16, 18, 20, 25, 30, 35, 40, 50, 60, 70, 80, 90, 100, 150, 200, 250 or more). These YPO0512 proteins include variants of SEQ ID NO:1. Preferred fragments of (b) comprise an epitope from SEQ ID NO:1. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:1. Other fragments omit one or more protein domains.

### (2) YPO0563

The ‘YPO0563’ sequence was annotated in reference 11 as ‘hypothetical protein’ (see GI:16120891). For reference purposes, the amino acid sequence of full-length YPO0563 as found in the Y. pestis CO92 strain is given as SEQ ID NO:3 herein. This protein is postulated herein to be a putative exported protein and furthermore to be a Secretion Monitor Precursor (SecM) protein.

Preferred YPO0563 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:3; and/or (b) that is a fragment of at least n consecutive amino acids of SEQ ID NO:3, wherein n is 7 or more (e.g. 8, 10, 12, 14, 16, 18, 20, 25, 30, 35, 40, 50, 60, 70, 80, 90, 100, 150, 200, 250 or more). These YPO0563 proteins include variants of SEQ ID NO:3. Preferred fragments of (b) comprise an epitope from SEQ ID NO:3. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:3. Other fragments omit one or more protein domains.

### (3) YPO3489

The ‘YPO3489’ sequence was annotated in reference 11 as ‘lipoprotein NlpI’ (see GI:16123635). For reference purposes, the amino acid sequence of full-length YPO3489 as found in the Y. pestis CO92 strain is given as SEQ ID NO:17 herein.

Preferred YPO3489 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:17; and/or (b) that is a fragment of at least n consecutive amino acids of SEQ ID NO:17, wherein n is 7 or more (e.g. 8, 10, 12, 14, 16, 18, 20, 25, 30, 35, 40, 50, 60, 70, 80, 90, 100, 150, 200, 250 or more). These YPO3489 proteins include variants of SEQ ID NO:17. Preferred fragments of (b) comprise an epitope from SEQ ID NO:17. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:17. Other fragments omit one or more protein domains.

### Second Antigen Group

### (4) YPO4003

The ‘YPO4003’ sequence was annotated in reference 11 as ‘periplasmic dipeptide transport protein’ (see GI:16124128), also known as dppA. For reference purposes, the amino acid sequence of full-length YPO4003 as found in the Y. pestis CO92 strain is given as SEQ ID NO:21 herein.

Preferred YPO4003 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:21; and/or (b) that is a fragment of at least n consecutive amino acids of SEQ ID NO:21, wherein n is 7 or more (e.g. 8, 10, 12, 14, 16, 18, 20, 25, 30, 35, 40, 50, 60, 70, 80, 90, 100, 150, 200, 250 or more). These YPO4003 proteins include variants of SEQ ID NO:21. Preferred fragments of (b) comprise an epitope from SEQ ID NO:21. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:21. Other fragments omit one or more protein domains.

### (5) YPO1604

The ‘YPO1604’ sequence was annotated in reference 11 as ‘hypothetical protein’ (see GI:16121872). For reference purposes, the amino acid sequence of full-length YPO1604 as found in the Y. pestis CO92 strain is given as SEQ ID NO:9 herein. This protein is postulated herein to be a putative exported protein.

Preferred YPO1604 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:9; and/or (b) that is a fragment of at least n consecutive amino acids of SEQ ID NO:9, wherein n is 7 or more (e.g. 8, 10, 12, 14, 16, 18, 20, 25, 30, 35, 40, 50, 60, 70, 80, 90, 100, 150, 200, 250 or more). These YPO1604 proteins include variants of SEQ ID NO:9. Preferred fragments of (b) comprise an epitope from SEQ ID NO:9. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:9. Other fragments omit one or more protein domains.

### Third Antigen Group

### (6) YPO3061

The ‘YPO3061’ sequence was annotated in reference 11 as ‘lipoprotein’ (see GI:16123238), also known as nlpB. For reference purposes, the amino acid sequence of full-length YPO3061 as found in the Y. pestis CO92 strain is given as SEQ ID NO:11 herein.

Preferred YPO3061 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:11; and/or (b) that is a fragment of at least n consecutive amino acids of SEQ ID NO:11, wherein n is 7 or more (e.g. 8, 10, 12, 14, 16, 18, 20, 25, 30, 35, 40, 50, 60, 70, 80, 90, 100, 150, 200, 250 or more). These YPO3061 proteins include variants of SEQ ID NO:11. Preferred fragments of (b) comprise an epitope from SEQ ID NO:11. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:11. Other fragments omit one or more protein domains.

### (7) YPO3559

The ‘YPO3559’ sequence was annotated in reference 11 as ‘hypothetical protein’ (see GI:16123703). For reference purposes, the amino acid sequence of full-length YPO3559 as found in the Y. pestis CO92 strain is given as SEQ ID NO:18 herein.

Preferred YPO3559 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:18; and/or (b) that is a fragment of at least n consecutive amino acids of SEQ ID NO:18, wherein n is 7 or more (e.g. 8, 10, 12, 14, 16, 18, 20, 25, 30, 35, 40, 50, 60, 70, 80, 90, 100, 150, 200, 250 or more). These YPO3559 proteins include variants of SEQ ID NO:18. Preferred fragments of (b) comprise an epitope from SEQ ID NO:18. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:18. Other fragments omit one or more protein domains.

### (8) YPO3382

The ‘YPO3382’ sequence was annotated in reference 11 as ‘global stress requirement protein GsrA’ (see GI:16123531), also known as htrA or degP. For reference purposes, the amino acid sequence of full-length YPO3382 as found in the Y. pestis CO92 strain is given as SEQ ID NO:15 herein.

Preferred YPO3382 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:15; and/or (b) that is a fragment of at least n consecutive amino acids of SEQ ID NO:15, wherein n is 7 or more (e.g. 8, 10, 12, 14, 16, 18, 20, 25, 30, 35, 40, 50, 60, 70, 80, 90, 100, 150, 200, 250 or more). These YPO3382 proteins include variants of SEQ ID NO:15. Preferred fragments of (b) comprise an epitope from SEQ ID NO:15. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:15. Other fragments omit one or more protein domains.

### (9) YPO0860

The ‘YPO0860’ sequence was annotated in reference 11 as ‘sugar-binding periplasmic protein’ (see GI:16121168). For reference purposes, the amino acid sequence of full-length YPO0860 as found in the Y. pestis CO92 strain is given as SEQ ID NO:5 herein.

Preferred YPO0860 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:5; and/or (b) that is a fragment of at least n consecutive amino acids of SEQ ID NO:5, wherein n is 7 or more (e.g. 8, 10, 12, 14, 16, 18, 20, 25, 30, 35, 40, 50, 60, 70, 80, 90, 100, 150, 200, 250 or more). These YPO0860 proteins include variants of SEQ ID NO:5. Preferred fragments of (b) comprise an epitope from SEQ ID NO:5. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:5. Other fragments omit one or more protein domains.

### (10) YPO0086

The ‘YPO0086’ sequence was annotated in reference 11 as ‘hypothetical protein’ (see GI:16120437). For reference purposes, the amino acid sequence of full-length YPO0086 as found in the Y. pestis CO92 strain is given as SEQ ID NO:2 herein. This protein is postulated herein to be a putative exported protein.

Preferred YPO0086 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:2; and/or (b) that is a fragment of at least n consecutive amino acids of SEQ ID NO:2, wherein n is 7 or more (e.g. 8, 10, 12, 14, 16, 18, 20, 25, 30, 35, 40, 50, 60, 70, 80, 90, 100, 150, 200, 250 or more). These YPO0086 proteins include variants of SEQ ID NO:2. Preferred fragments of (b) comprise an epitope from SEQ ID NO:2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:2. Other fragments omit one or more protein domains.

### (11) YPO3631

The ‘YPO3631’ sequence was annotated in reference 11 as ‘hypothetical protein’ (see GI:16123773). For reference purposes, the amino acid sequence of full-length YPO3631 as found in the Y. pestis CO92 strain is given as SEQ ID NO:19 herein. This protein is postulated herein to be a putative exported protein.

Preferred YPO3631 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:19; and/or (b) that is a fragment of at least n consecutive amino acids of SEQ ID NO:19, wherein n is 7 or more (e.g. 8, 10, 12, 14, 16, 18, 20, 25, 30, 35, 40, 50, 60, 70, 80, 90, 100, 150, 200, 250 or more). These YPO3631 proteins include variants of SEQ ID NO:19. Preferred fragments of (b) comprise an epitope from SEQ ID NO:19. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:19. Other fragments omit one or more protein domains.

### (12) YPO2881

The ‘YPO2881’ sequence was annotated in reference 11 as ‘putative fimbrial biogenesis protein’ (see GI:16123073). For reference purposes, the amino acid sequence of full-length YPO2881 as found in the Y. pestis CO92 strain is given as SEQ ID NO:10 herein.

Preferred YPO2881 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:10; and/or (b) that is a fragment of at least n consecutive amino acids of SEQ ID NO:10, wherein n is 7 or more (e.g. 8, 10, 12, 14, 16, 18, 20, 25, 30, 35, 40, 50, 60, 70, 80, 90, 100, 150, 200, 250 or more). These YPO2881 proteins include variants of SEQ ID NO:10. Preferred fragments of (b) comprise an epitope from SEQ ID NO:10. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:10. Other fragments omit one or more protein domains.

### (13) YPO3343

The ‘YPO3343’ sequence was annotated in reference 11 as ‘probable extracellular solute-binding protein’ (see GI:16123493). For reference purposes, the amino acid sequence of full-length YPO3343 as found in the Y. pestis CO92 strain is given as SEQ ID NO:13 herein.

Preferred YPO3343 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:13; and/or (b) that is a fragment of at least n consecutive amino acids of SEQ ID NO:13, wherein n is 7 or more (e.g. 8, 10, 12, 14, 16, 18, 20, 25, 30, 35, 40, 50, 60, 70, 80, 90, 100, 150, 200, 250 or more). These YPO3343 proteins include variants of SEQ ID NO:13. Preferred fragments of (b) comprise an epitope from SEQ ID NO:13. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:13. Other fragments omit one or more protein domains.

### (14) YPO3361

The ‘YPO3361’ sequence was annotated in reference 11 as ‘4-diphosphocytidyl-2C-methyl-D-erythritol synthase’ (see GI:16123511). For reference purposes, the amino acid sequence of full-length YPO3361 as found in the Y. pestis CO92 strain is given as SEQ ID NO:14 herein.

Preferred YPO3361 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:14; and/or (b) that is a fragment of at least n consecutive amino acids of SEQ ID NO:14, wherein n is 7 or more (e.g. 8, 10, 12, 14, 16, 18, 20, 25, 30, 35, 40, 50, 60, 70, 80, 90, 100, 150, 200, 250 or more). These YPO3361 proteins include variants of SEQ ID NO:14. Preferred fragments of (b) comprise an epitope from SEQ ID NO:14. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:14. Other fragments omit one or more protein domains.

### (15) YPO3430

The ‘YPO3430’ sequence was annotated in reference 11 as ‘hypothetical protein’ (see GI:16123579). For reference purposes, the amino acid sequence of full-length YPO3430 as found in the Y. pestis CO92 strain is given as SEQ ID NO:16 herein.

Preferred YPO3430 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:16; and/or (b) that is a fragment of at least n consecutive amino acids of SEQ ID NO:16, wherein n is 7 or more (e.g. 8, 10, 12, 14, 16, 18, 20, 25, 30, 35, 40, 50, 60, 70, 80, 90, 100, 150, 200, 250 or more). These YPO3430 proteins include variants of SEQ ID NO:16. Preferred fragments of (b) comprise an epitope from SEQ ID NO:16. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:16. Other fragments omit one or more protein domains.

### (16) YPO1411

The ‘YPO1411’ sequence was annotated in reference 11 as ‘putative outer membrane porin C protein’ (see GI:16121691). For reference purposes, the amino acid sequence of full-length YPO1411 as found in the Y. pestis CO92 strain is given as SEQ ID NO:8 herein.

Preferred YPO1411 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:8; and/or (b) that is a fragment of at least n consecutive amino acids of SEQ ID NO:8, wherein n is 7 or more (e.g. 8, 10, 12, 14, 16, 18, 20, 25, 30, 35, 40, 50, 60, 70, 80, 90, 100, 150, 200, 250 or more). These YPO1411 proteins include variants of SEQ ID NO:8. Preferred fragments of (b) comprise an epitope from SEQ ID NO:8. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:8. Other fragments omit one or more protein domains.

### (17) YPO3935

The ‘YPO3935’ sequence was annotated in reference 11 as ‘membrane protein’ (see GI:16124063). For reference purposes, the amino acid sequence of full-length YPO3935 as found in the Y. pestis CO92 strain is given as SEQ ID NO:20 herein.

Preferred YPO3935 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:20; and/or (b) that is a fragment of at least n consecutive amino acids of SEQ ID NO:20, wherein n is 7 or more (e.g. 8, 10, 12, 14, 16, 18, 20, 25, 30, 35, 40, 50, 60, 70, 80, 90, 100, 150, 200, 250 or more). These YPO3935 proteins include variants of SEQ ID NO:20. Preferred fragments of (b) comprise an epitope from SEQ ID NO:20. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:20. Other fragments omit one or more protein domains.

### (18) YPO0809

The ‘YPO0809’ sequence was annotated in reference 11 as ‘general secretion pathway protein K’ (see GI:16121121). For reference purposes, the amino acid sequence of full-length YPO0809 as found in the Y. pestis CO92 strain is given as SEQ ID NO:4 herein.

Preferred YPO0809 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:4; and/or (b) that is a fragment of at least n consecutive amino acids of SEQ ID NO:4, wherein n is 7 or more (e.g. 8, 10, 12, 14, 16, 18, 20, 25, 30, 35, 40, 50, 60, 70, 80, 90, 100, 150, 200, 250 or more). These YPO0809 proteins include variants of SEQ ID NO:4. Preferred fragments of (b) comprise an epitope from SEQ ID NO:4. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:4. Other fragments omit one or more protein domains.

### (19) YPO1123

The ‘YPO1123’ sequence was annotated in reference 11 as ‘TolA colicin import membrane protein’ (see GI:16121423). For reference purposes, the amino acid sequence of full-length YPO1123 as found in the Y. pestis CO92 strain is given as SEQ ID NO:7 herein.

Preferred YPO1123 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:7; and/or (b) that is a fragment of at least n consecutive amino acids of SEQ ID NO:7, wherein n is 7 or more (e.g. 8, 10, 12, 14, 16, 18, 20, 25, 30, 35, 40, 50, 60, 70, 80, 90, 100, 150, 200, 250 or more). These YPO1123 proteins include variants of SEQ ID NO:7. Preferred fragments of (b) comprise an epitope from SEQ ID NO:7. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:7. Other fragments omit one or more protein domains.

### (20) YPO3065

The ‘YPO3065’ sequence was annotated in reference 11 as ‘hypothetical protein’ (see GI:16123242). For reference purposes, the amino acid sequence of full-length YPO3065 as found in the Y. pestis CO92 strain is given as SEQ ID NO:12 herein.

Preferred YPO3065 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:12; and/or (b) that is a fragment of at least n consecutive amino acids of SEQ ID NO:12, wherein n is 7 or more (e.g. 8, 10, 12, 14, 16, 18, 20, 25, 30, 35, 40, 50, 60, 70, 80, 90, 100, 150, 200, 250 or more). These YPO3065 proteins include variants of SEQ ID NO:12. Preferred fragments of (b) comprise an epitope from SEQ ID NO:12. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:12. Other fragments omit one or more protein domains.

### (21) YPO1070

The ‘YPO1070’ sequence was annotated in reference 11 as ‘putative lipoprotein’ (see GI:16121371), also known as rcsF. For reference purposes, the amino acid sequence of full-length YPO1070 as found in the Y. pestis CO92 strain is given as SEQ ID NO:6 herein.

Preferred YPO1070 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:6; and/or (b) that is a fragment of at least n consecutive amino acids of SEQ ID NO:6, wherein n is 7 or more (e.g. 8, 10, 12, 14, 16, 18, 20, 25, 30, 35, 40, 50, 60, 70, 80, 90, 100, 150, 200, 250 or more). These YPO1070 proteins include variants of SEQ ID NO:6. Preferred fragments of (b) comprise an epitope from SEQ ID NO:6. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:6. Other fragments omit one or more protein domains.

### Fourth Antigen Group

### (1) YPO0102

The ‘YPO0102’ sequence was annotated in reference 11 as ‘hypothetical protein’ (see GI:16120449). For reference purposes, the amino acid sequence of full-length YPO0102 as found in the Y. pestis CO92 strain is given as SEQ ID NO:44 herein.

Preferred YPO0102 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:44; and/or (b) that is a fragment of at least n consecutive amino acids of SEQ ID NO:44, wherein n is 7 or more (e.g. 8, 10, 12, 14, 16, 18, 20, 25, 30, 35, 40, 50, 60, 70, 80, 90, 100, 150, 200, 250 or more). These YPO0102 proteins include variants of SEQ ID NO:44. Preferred fragments of (b) comprise an epitope from SEQ ID NO:44. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:44. Other fragments omit one or more protein domains.

### (2) YPO0570

The ‘YPO0570’ sequence was annotated in reference 11 as ‘putative membrane protein’ (see GI:16120899). For reference purposes, the amino acid sequence of full-length YPO0570 as found in the Y. pestis CO92 strain is given as SEQ ID NO:35 herein.

Preferred YPO0570 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:35; and/or (b) that is a fragment of at least n consecutive amino acids of SEQ ID NO:35, wherein n is 7 or more (e.g. 8, 10, 12, 14, 16, 18, 20, 25, 30, 35, 40, 50, 60, 70, 80, 90, 100, 150, 200, 250 or more). These YPO0570 proteins include variants of SEQ ID NO:35. Preferred fragments of (b) comprise an epitope from SEQ ID NO:35. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:35. Other fragments omit one or more protein domains.

### (3) YPO1053

The ‘YPO1053’ sequence was annotated in reference 11 as ‘cationic 19 kDa outer membrane protein precursor’ (see GI:16121353). For reference purposes, the amino acid sequence of full-length YPO1053 as found in the Y. pestis CO92 strain is given as SEQ ID NO:33 herein. This protein is postulated herein to be a member of the OmpH family of proteins.

Preferred YPO1053 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:33; and/or (b) that is a fragment of at least n consecutive amino acids of SEQ ID NO:33, wherein n is 7 or more (e.g. 8, 10, 12, 14, 16, 18, 20, 25, 30, 35, 40, 50, 60, 70, 80, 90, 100, 150, 200, 250 or more). These YPO1053 proteins include variants of SEQ ID NO:33. Preferred fragments of (b) comprise an epitope from SEQ ID NO:33. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:33. Other fragments omit one or more protein domains.

### (4) YPO1435

The ‘YPO1435’ sequence was annotated in reference 11 as ‘putative outer membrane porin A protein’ (see GI:16121713). For reference purposes, the amino acid sequence of full-length YPO1435 as found in the Y. pestis CO92 strain is given as SEQ ID NO:32 herein.

Preferred YPO1435 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:32; and/or (b) that is a fragment of at least n consecutive amino acids of SEQ ID NO:32, wherein n is 7 or more (e.g. 8, 10, 12, 14, 16, 18, 20, 25, 30, 35, 40, 50, 60, 70, 80, 90, 100, 150, 200, 250 or more). These YPO1435 proteins include variants of SEQ ID NO:32. Preferred fragments of (b) comprise an epitope from SEQ ID NO:32. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:32. Other fragments omit one or more protein domains.

### (5) YPO2674

The ‘YPO2674’ sequence was annotated in reference 11 as ‘hypothetical protein’ (see GI:16122879). For reference purposes, the amino acid sequence of full-length YPO2674 as found in the Y. pestis CO92 strain is given as SEQ ID NO:26 herein.

Preferred YPO2674 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:26; and/or (b) that is a fragment of at least n consecutive amino acids of SEQ ID NO:26, wherein n is 7 or more (e.g. 8, 10, 12, 14, 16, 18, 20, 25, 30, 35, 40, 50, 60, 70, 80, 90, 100, 150, 200, 250 or more). These YPO2674 proteins include variants of SEQ ID NO:26. Preferred fragments of (b) comprise an epitope from SEQ ID NO:26. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:26. Other fragments omit one or more protein domains.

### (6) YPO2292

The ‘YPO2292’ sequence was annotated in reference 11 as ‘putative lipoprotein’ (see GI:16122516). For reference purposes, the amino acid sequence of full-length YPO2292 as found in the Y. pestis CO92 strain is given as SEQ ID NO:29 herein.

Preferred YPO2292 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:29; and/or (b) that is a fragment of at least n consecutive amino acids of SEQ ID NO:29, wherein n is 7 or more (e.g. 8, 10, 12, 14, 16, 18, 20, 25, 30, 35, 40, 50, 60, 70, 80, 90, 100, 150, 200, 250 or more). These YPO2292 proteins include variants of SEQ ID NO:29. Preferred fragments of (b) comprise an epitope from SEQ ID NO:29. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:29. Other fragments omit one or more protein domains.

### (7) YPO3050

The ‘YPO3050’ sequence was annotated in reference 11 as ‘hypothetical protein’ (see GI:16123227). For reference purposes, the amino acid sequence of full-length YPO3050 as found in the Y. pestis CO92 strain is given as SEQ ID NO:25 herein.

Preferred YPO3050 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:25; and/or (b) that is a fragment of at least n consecutive amino acids of SEQ ID NO:25, wherein n is 7 or more (e.g. 8, 10, 12, 14, 16, 18, 20, 25, 30, 35, 40, 50, 60, 70, 80, 90, 100, 150, 200, 250 or more). These YPO3050 proteins include variants of SEQ ID NO:25. Preferred fragments of (b) comprise an epitope from SEQ ID NO:25. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:25. Other fragments omit one or more protein domains.

### (8) YPO2615

The ‘YPO2615’ sequence was annotated in reference 11 as ‘putative amino acid-binding protein precursor’ (see GI:16122828). For reference purposes, the amino acid sequence of full-length YPO2615 as found in the Y. pestis CO92 strain is given as SEQ ID NO:27 herein.

Preferred YPO2615 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:27; and/or (b) that is a fragment of at least n consecutive amino acids of SEQ ID NO:27, wherein n is 7 or more (e.g. 8, 10, 12, 14, 16, 18, 20, 25, 30, 35, 40, 50, 60, 70, 80, 90, 100, 150, 200, 250 or more). These YPO2615 proteins include variants of SEQ ID NO:27. Preferred fragments of (b) comprise an epitope from SEQ ID NO:27. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:27. Other fragments omit one or more protein domains.

### (9) YPO1507

The ‘YPO1507’ sequence was annotated in reference 11 as ‘galactose-binding protein’ (see GI:16121780). For reference purposes, the amino acid sequence of full-length YPO1507 as found in the Y. pestis CO92 strain is given as SEQ ID NO:31 herein.

Preferred YPO1507 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:31; and/or (b) that is a fragment of at least n consecutive amino acids of SEQ ID NO:31, wherein n is 7 or more (e.g. 8, 10, 12, 14, 16, 18, 20, 25, 30, 35, 40, 50, 60, 70, 80, 90, 100, 150, 200, 250 or more). These YPO1507 proteins include variants of SEQ ID NO:31. Preferred fragments of (b) comprise an epitope from SEQ ID NO:31. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:31. Other fragments omit one or more protein domains.

### (10) YPO4111

The ‘YPO4111’ sequence was annotated in reference 11 as ‘putative periplasmic solute-binding protein’ (see GI:16124219). For reference purposes, the amino acid sequence of full-length YPO4111 as found in the Y. pestis CO92 strain is given as SEQ ID NO:47 herein.

Preferred YPO4111 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:47; and/or (b) that is a fragment of at least n consecutive amino acids of SEQ ID NO:47, wherein n is 7 or more (e.g. 8, 10, 12, 14, 16, 18, 20, 25, 30, 35, 40, 50, 60, 70, 80, 90, 100, 150, 200, 250 or more). These YPO4111 proteins include variants of SEQ ID NO:47. Preferred fragments of (b) comprise an epitope from SEQ ID NO:47. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:47. Other fragments omit one or more protein domains.

### (11) YPO0015

The ‘YPO0015’ sequence was annotated in reference 11 as ‘secreted thiol:disulfide interchange protein DsbA’ (see GI:16120369). For reference purposes, the amino acid sequence of full-length YPO0015 as found in the Y. pestis CO92 strain is given as SEQ ID NO:46 herein.

Preferred YPO0015 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:46; and/or (b) that is a fragment of at least n consecutive amino acids of SEQ ID NO:46, wherein n is 7 or more (e.g. 8, 10, 12, 14, 16, 18, 20, 25, 30, 35, 40, 50, 60, 70, 80, 90, 100, 150, 200, 250 or more). These YPO0015 proteins include variants of SEQ ID NO:46. Preferred fragments of (b) comprise an epitope from SEQ ID NO:46. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:46. Other fragments omit one or more protein domains.

### (12) YPO0195

The ‘YPO0195’ sequence was annotated in reference 11 as ‘peptidyl-prolyl cis-trans isomerase’ (see GI:16120534). For reference purposes, the amino acid sequence of full-length YPO0195 as found in the Y. pestis CO92 strain is given as SEQ ID NO:43 herein.

Preferred YPO0195 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:43; and/or (b) that is a fragment of at least n consecutive amino acids of SEQ ID NO:43, wherein n is 7 or more (e.g. 8, 10, 12, 14, 16, 18, 20, 25, 30, 35, 40, 50, 60, 70, 80, 90, 100, 150, 200, 250 or more). These YPO0195 proteins include variants of SEQ ID NO:43. Preferred fragments of (b) comprise an epitope from SEQ ID NO:43. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:43. Other fragments omit one or more protein domains.

### (13) YPO2342

The ‘YPO2342’ sequence was annotated in reference 11 as ‘thiol peroxidase’ (see GI:16122566). For reference purposes, the amino acid sequence of full-length YPO2342 as found in the Y. pestis CO92 strain is given as SEQ ID NO:28 herein.

Preferred YPO2342 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:28; and/or (b) that is a fragment of at least n consecutive amino acids of SEQ ID NO:28, wherein n is 7 or more (e.g. 8, 10, 12, 14, 16, 18, 20, 25, 30, 35, 40, 50, 60, 70, 80, 90, 100, 150, 200, 250 or more). These YPO2342 proteins include variants of SEQ ID NO:28. Preferred fragments of (b) comprise an epitope from SEQ ID NO:28. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:28. Other fragments omit one or more protein domains.

### (14) YPO0501

The ‘YPO0501’ sequence was annotated in reference 11 as ‘hypothetical protein’ (see GI:16120831). For reference purposes, the amino acid sequence of full-length YPO0501 as found in the Y. pestis CO92 strain is given as SEQ ID NO:37 herein. However, it is postulated herein that YPO0501 forms part of a Type Three Secretion System (TTSS).

Preferred YPO0501 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:37; and/or (b) that is a fragment of at least n consecutive amino acids of SEQ ID NO:37, wherein n is 7 or more (e.g. 8, 10, 12, 14, 16, 18, 20, 25, 30, 35, 40, 50, 60, 70, 80, 90, 100, 150, 200, 250 or more). These YPO0501 proteins include variants of SEQ ID NO:37. Preferred fragments of (b) comprise an epitope from SEQ ID NO:37. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:37. Other fragments omit one or more protein domains.

### (15) YPO0502

The ‘YPO0502’ sequence was annotated in reference 11 as ‘hypothetical protein’ (see GI:16120832). For reference purposes, the amino acid sequence of full-length YPO0502 as found in the Y. pestis CO92 strain is given as SEQ ID NO:36 herein. However, it is postulated herein that YPO0502 forms part of a Type Three Secretion System (TTSS).

Preferred YPO0502 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:36; and/or (b) that is a fragment of at least n consecutive amino acids of SEQ ID NO:36, wherein n is 7 or more (e.g. 8, 10, 12, 14, 16, 18, 20, 25, 30, 35, 40, 50, 60, 70, 80, 90, 100, 150, 200, 250 or more). These YPO0502 proteins include variants of SEQ ID NO:36. Preferred fragments of (b) comprise an epitope from SEQ ID NO:36. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:36. Other fragments omit one or more protein domains.

### (16) YPO0819

The ‘YPO0819’ sequence was annotated in reference 11 as ‘putative carbonic anhydrase’ (see GI:16121130). For reference purposes, the amino acid sequence of full-length YPO0819 as found in the Y. pestis CO92 strain is given as SEQ ID NO:34 herein.

Preferred YPO0819 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:34; and/or (b) that is a fragment of at least n consecutive amino acids of SEQ ID NO:34, wherein n is 7 or more (e.g. 8, 10, 12, 14, 16, 18, 20, 25, 30, 35, 40, 50, 60, 70, 80, 90, 100, 150, 200, 250 or more). These YPO0819 proteins include variants of SEQ ID NO:34. Preferred fragments of (b) comprise an epitope from SEQ ID NO:34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:34. Other fragments omit one or more protein domains.

### (17) YPO3644

The ‘YPO3644’ sequence was annotated in reference 11 as ‘major cold shock protein Cspa1’ (see GI:16123786). For reference purposes, the amino acid sequence of full-length YPO3644 as found in the Y. pestis CO92 strain is given as SEQ ID NO:22 herein.

Preferred YPO3644 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:22; and/or (b) that is a fragment of at least n consecutive amino acids of SEQ ID NO:22, wherein n is 7 or more (e.g. 8, 10, 12, 14, 16, 18, 20, 25, 30, 35, 40, 50, 60, 70, 80, 90, 100, 150, 200, 250 or more). These YPO3644 proteins include variants of SEQ ID NO:22. Preferred fragments of (b) comprise an epitope from SEQ ID NO:22. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:22. Other fragments omit one or more protein domains.

### (18) YPO1746

The ‘YPO1746’ sequence was annotated in reference 11 as ‘cold shock protein’ (see GI:16122003). For reference purposes, the amino acid sequence of full-length YPO1746 as found in the Y. pestis CO92 strain is given as SEQ ID NO:30 herein.

Preferred YPO1746 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:30; and/or (b) that is a fragment of at least n consecutive amino acids of SEQ ID NO:30, wherein n is 7 or more (e.g. 8, 10, 12, 14, 16, 18, 20, 25, 30, 35, 40, 50, 60, 70, 80, 90, 100, 150, 200, 250 or more). These YPO1746 proteins include variants of SEQ ID NO:30. Preferred fragments of (b) comprise an epitope from SEQ ID NO:30. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:30. Other fragments omit one or more protein domains.

### (19) YPO0351

The ‘YPO0351’ sequence was annotated in reference 11 as ‘60 kDa chaperonin’ (see GI:16120686). For reference purposes, the amino acid sequence of full-length YPO0351 as found in the Y. pestis CO92 strain is given as SEQ ID NO:39 herein.

Preferred YPO0351 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:39; and/or (b) that is a fragment of at least n consecutive amino acids of SEQ ID NO:39, wherein n is 7 or more (e.g. 8, 10, 12, 14, 16, 18, 20, 25, 30, 35, 40, 50, 60, 70, 80, 90, 100, 150, 200, 250 or more). These YPO0351 proteins include variants of SEQ ID NO:39. Preferred fragments of (b) comprise an epitope from SEQ ID NO:39. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:39. Other fragments omit one or more protein domains. A YPO0351 antigen has been shown to be an outer membrane protein suitable for use as an antigenic protein in reference 12.

### (20) YPO0468

The ‘YPO0468’ sequence was annotated in reference 11 as ‘chaperone protein DnaK’ (see GI:16120797). For reference purposes, the amino acid sequence of full-length YPO0468 as found in the Y. pestis CO92 strain is given as SEQ ID NO:38 herein.

Preferred YPO0468 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:38; and/or (b) that is a fragment of at least n consecutive amino acids of SEQ ID NO:38, wherein n is 7 or more (e.g. 8, 10, 12, 14, 16, 18, 20, 25, 30, 35, 40, 50, 60, 70, 80, 90, 100, 150, 200, 250 or more). These YPO0468 proteins include variants of SEQ ID NO:38. Preferred fragments of (b) comprise an epitope from SEQ ID NO:38. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:38. Other fragments omit one or more protein domains. A YPO0468 antigen has been shown to be an outer membrane protein suitable for use as an antigenic protein in reference 12.

### (21) YPO0203

The ‘YPO0203’ sequence was annotated in reference 11 as ‘elongation factor Tu’ (see GI:16120542). For reference purposes, the amino acid sequence of full-length YPO0203 as found in the Y. pestis CO92 strain is given as SEQ ID NO:42 herein.

Preferred YPO0203 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:42; and/or (b) that is a fragment of at least n consecutive amino acids of SEQ ID NO:42, wherein n is 7 or more (e.g. 8, 10, 12, 14, 16, 18, 20, 25, 30, 35, 40, 50, 60, 70, 80, 90, 100, 150, 200, 250 or more). These YPO0203 proteins include variants of SEQ ID NO:42. Preferred fragments of (b) comprise an epitope from SEQ ID NO:42. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:42. Other fragments omit one or more protein domains. A YPO0203 antigen has been shown to be an outer membrane protein suitable for use as an antigenic protein in reference 12.

### (22) YPO0216

The ‘YPO0216’ sequence was annotated in reference 11 as ‘30S ribosomal protein S3’ (see GI:16120553). For reference purposes, the amino acid sequence of full-length YPO0216 as found in the Y. pestis CO92 strain is given as SEQ ID NO:41 herein.

Preferred YPO0216 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:41; and/or (b) that is a fragment of at least n consecutive amino acids of SEQ ID NO:41, wherein n is 7 or more (e.g. 8, 10, 12, 14, 16, 18, 20, 25, 30, 35, 40, 50, 60, 70, 80, 90, 100, 150, 200, 250 or more). These YPO0216 proteins include variants of SEQ ID NO:41. Preferred fragments of (b) comprise an epitope from SEQ ID NO:41. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:41. Other fragments omit one or more protein domains.

### (23) YPO3536

The ‘YPO3536’ sequence was annotated in reference 11 as ‘50S ribosomal protein L9’ (see GI:16123682). For reference purposes, the amino acid sequence of full-length YPO3536 as found in the Y. pestis CO92 strain is given as SEQ ID NO:24 herein.

Preferred YPO3536 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:24; and/or (b) that is a fragment of at least n consecutive amino acids of SEQ ID NO:24, wherein n is 7 or more (e.g. 8, 10, 12, 14, 16, 18, 20, 25, 30, 35, 40, 50, 60, 70, 80, 90, 100, 150, 200, 250 or more). These YPO3536 proteins include variants of SEQ ID NO:24. Preferred fragments of (b) comprise an epitope from SEQ ID NO:24. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:24. Other fragments omit one or more protein domains.

### (24) YPO0233

The ‘YPO0233’ sequence was annotated in reference 11 as ‘30S ribosomal protein S4’ (see GI:16120571). For reference purposes, the amino acid sequence of full-length YPO0233 as found in the Y. pestis CO92 strain is given as SEQ ID NO:40 herein.

Preferred YPO0233 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:40; and/or (b) that is a fragment of at least n consecutive amino acids of SEQ ID NO:40, wherein n is 7 or more (e.g. 8, 10, 12, 14, 16, 18, 20, 25, 30, 35, 40, 50, 60, 70, 80, 90, 100, 150, 200, 250 or more). These YPO0233 proteins include variants of SEQ ID NO:40. Preferred fragments of (b) comprise an epitope from SEQ ID NO:40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:40. Other fragments omit one or more protein domains.

### (25) YPO0067

The ‘YPO0067’ sequence was annotated in reference 11 as ‘protein-export protein’ (see GI:16120418). For reference purposes, the amino acid sequence of full-length YPO0067 as found in the Y. pestis CO92 strain is given as SEQ ID NO:45 herein.

Preferred YPO0067 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:45; and/or (b) that is a fragment of at least n consecutive amino acids of SEQ ID NO:45, wherein n is 7 or more (e.g. 8, 10, 12, 14, 16, 18, 20, 25, 30, 35, 40, 50, 60, 70, 80, 90, 100, 150, 200, 250 or more). These YPO0067 proteins include variants of SEQ ID NO:45. Preferred fragments of (b) comprise an epitope from SEQ ID NO:45. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:45. Other fragments omit one or more protein domains.

### (26) YPO03643

The ‘YPO3643’ sequence was annotated in reference 11 as ‘major cold shock protein Cspa2’ (see GI:16123785). For reference purposes, the amino acid sequence of full-length YPO3643 as found in the Y. pestis CO92 strain is given as SEQ ID NO:23 herein.

Preferred YPO3643 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:23; and/or (b) that is a fragment of at least n consecutive amino acids of SEQ ID NO:23, wherein n is 7 or more (e.g. 8, 10, 12, 14, 16, 18, 20, 25, 30, 35, 40, 50, 60, 70, 80, 90, 100, 150, 200, 250 or more). These YPO3643 proteins include variants of SEQ ID NO:23. Preferred fragments of (b) comprise an epitope from SEQ ID NO:23. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:23. Other fragments omit one or more protein domains.

### (27) YPO3375

The ‘YPO3375’ sequence was annotated in reference 11 as ‘superoxide dismutase [Cu—Zn] precursor’ (see GI:16123524). For reference purposes, the amino acid sequence of full-length YPO3375 as found in the Y. pestis CO92 strain is given as SEQ ID NO:58 herein.

Preferred YPO3375 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:58; and/or (b) that is a fragment of at least n consecutive amino acids of SEQ ID NO:58, wherein n is 7 or more (e.g. 8, 10, 12, 14, 16, 18, 20, 25, 30, 35, 40, 50, 60, 70, 80, 90, 100, 150, 200, 250 or more). These YPO3375 proteins include variants of SEQ ID NO:58. Preferred fragments of (b) comprise an epitope from SEQ ID NO:58. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:58. Other fragments omit one or more protein domains.

### (28) YPO0494

The ‘YPO0494’ sequence was annotated in reference 11 as ‘survival protein SurA precursor (peptidyl-prolyl cis-trans isomerase’ (see GI:16120824). For reference purposes, the amino acid sequence of full-length YPO0494 as found in the Y. pestis CO92 strain is given as SEQ ID NO:53 herein.

Preferred YPO0494 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:53; and/or (b) that is a fragment of at least n consecutive amino acids of SEQ ID NO:53, wherein n is 7 or more (e.g. 8, 10, 12, 14, 16, 18, 20, 25, 30, 35, 40, 50, 60, 70, 80, 90, 100, 150, 200, 250 or more). These YPO0494 proteins include variants of SEQ ID NO:53. Preferred fragments of (b) comprise an epitope from SEQ ID NO:53. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:53. Other fragments omit one or more protein domains.

### (29) YPO1052

The ‘YPO1052’ sequence was annotated in reference 11 as ‘ putative surface antigen’ (see GI:16121352). For reference purposes, the amino acid sequence of full-length YPO1052 as found in the Y. pestis CO92 strain is given as SEQ ID NO:51 herein.

Preferred YPO1052 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:51; and/or (b) that is a fragment of at least n consecutive amino acids of SEQ ID NO:51, wherein n is 7 or more (e.g. 8, 10, 12, 14, 16, 18, 20, 25, 30, 35, 40, 50, 60, 70, 80, 90, 100, 150, 200, 250 or more). These YPO1052 proteins include variants of SEQ ID NO:51. Preferred fragments of (b) comprise an epitope from SEQ ID NO:51. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:51. Other fragments omit one or more protein domains.

### (30) YPO1906

The ‘YPO1906’ sequence was annotated in reference 11 as ‘pesticin/yersiniabactin receptor protein’ (see GI:16122154). For reference purposes, the amino acid sequence of full-length YPO1906 as found in the Y. pestis CO92 strain is given as SEQ ID NO:56 herein.

Preferred YPO1906 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:56; and/or (b) that is a fragment of at least n consecutive amino acids of SEQ ID NO:56, wherein n is 7 or more (e.g. 8, 10, 12, 14, 16, 18, 20, 25, 30, 35, 40, 50, 60, 70, 80, 90, 100, 150, 200, 250 or more). These YPO1906 proteins include variants of SEQ ID NO:56. Preferred fragments of (b) comprise an epitope from SEQ ID NO:56. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:56. Other fragments omit one or more protein domains.

### (31) YPO0663

The ‘YPO0663’ sequence was annotated in reference 11 as ‘ABC-transporter outer membrane component’ (see GI:16120988). For reference purposes, the amino acid sequence of full-length YPO0663 as found in the Y. pestis CO92 strain is given as SEQ ID NO:54 herein.

Preferred YPO0663 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:54; and/or (b) that is a fragment of at least n consecutive amino acids of SEQ ID NO:54, wherein n is 7 or more (e.g. 8, 10, 12, 14, 16, 18, 20, 25, 30, 35, 40, 50, 60, 70, 80, 90, 100, 150, 200, 250 or more). These YPO0663 proteins include variants of SEQ ID NO:54. Preferred fragments of (b) comprise an epitope from SEQ ID NO:54. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:54. Other fragments omit one or more protein domains.

### (32) YPO1222

The ‘YPO1222’ sequence was annotated in reference 11 as ‘outer membrane protein C, porin’ (see GI:16121511). For reference purposes, the amino acid sequence of full-length YPO1222 as found in the Y. pestis CO92 strain is given as SEQ ID NO:55 herein.

Preferred YPO1222 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:55; and/or (b) that is a fragment of at least n consecutive amino acids of SEQ ID NO:55, wherein n is 7 or more (e.g. 8, 10, 12, 14, 16, 18, 20, 25, 30, 35, 40, 50, 60, 70, 80, 90, 100, 150, 200, 250 or more). These YPO1222 proteins include variants of SEQ ID NO:55. Preferred fragments of (b) comprise an epitope from SEQ ID NO:55. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:55. Other fragments omit one or more protein domains. A YPO1222 antigen has been shown to be an outer membrane protein suitable for use as an antigenic protein in reference 12.

### (33) YPO2905

The ‘YPO2905’ sequence was annotated in reference 11 as ‘attachment invasion locus protein’ (see GI:16123096). For reference purposes, the amino acid sequence of full-length YPO2905 as found in the Y. pestis CO92 strain is given as SEQ ID NO:57 herein.

Preferred YPO2905 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:57; and/or (b) that is a fragment of at least n consecutive amino acids of SEQ ID NO:57, wherein n is 7 or more (e.g. 8, 10, 12, 14, 16, 18, 20, 25, 30, 35, 40, 50, 60, 70, 80, 90, 100, 150, 200, 250 or more). These YPO2905 proteins include variants of SEQ ID NO:57. Preferred fragments of (b) comprise an epitope from SEQ ID NO:57. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:57. Other fragments omit one or more protein domains.

### (34) YPO4070

The ‘YPO4070’ sequence was annotated in reference 11 as ‘hypothetical protein’ (see GI:16124183). For reference purposes, the amino acid sequence of full-length YPO4070 as found in the Y. pestis CO92 strain is given as SEQ ID NO:52 herein.

Preferred YPO4070 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:52; and/or (b) that is a fragment of at least n consecutive amino acids of SEQ ID NO:52, wherein n is 7 or more (e.g. 8, 10, 12, 14, 16, 18, 20, 25, 30, 35, 40, 50, 60, 70, 80, 90, 100, 150, 200, 250 or more). These YPO4070 proteins include variants of SEQ ID NO:52. Preferred fragments of (b) comprise an epitope from SEQ ID NO:52. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:52. Other fragments omit one or more protein domains.

### (35) YPPCP1.07

The ‘YPPCP1.07’ sequence was annotated in reference 11 as ‘plasminogen activator protease precursor’ (see GI:16082686). For reference purposes, the amino acid sequence of full-length YPPCP1.07 as found in the Y. pestis CO92 strain is given as SEQ ID NO:50 herein.

Preferred YPPCP1.07 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:50; and/or (b) that is a fragment of at least n consecutive amino acids of SEQ ID NO:50, wherein n is 7 or more (e.g. 8, 10, 12, 14, 16, 18, 20, 25, 30, 35, 40, 50, 60, 70, 80, 90, 100, 150, 200, 250 or more). These YPPCP1.07 proteins include variants (e.g. allelic variants, polymorphic forms, homologs, orthologs, paralogs, mutants, etc.) of SEQ ID NO:50. Preferred fragments of (b) comprise an epitope from SEQ ID NO:50. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:50. Other fragments omit one or more protein domains.

### (36) YPMT1.42

The ‘YPMT1.42’ sequence was annotated in reference 11 as ‘putative periplasmic protein’ (see GI:16082828). For reference purposes, the amino acid sequence of full-length YPMT1.42 as found in the Y. pestis CO92 strain is given as SEQ ID NO:59 herein.

Preferred YPMT1.42 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:59; and/or (b) that is a fragment of at least n consecutive amino acids of SEQ ID NO:59, wherein n is 7 or more (e.g. 8, 10, 12, 14, 16, 18, 20, 25, 30, 35, 40, 50, 60, 70, 80, 90, 100, 150, 200, 250 or more). These YPMT1.42 proteins include variants (e.g. allelic variants, polymorphic forms, homologs, orthologs, paralogs, mutants, etc.) of SEQ ID NO:59. Preferred fragments of (b) comprise an epitope from SEQ ID NO:59. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:59. Other fragments omit one or more protein domains.

### Fifth Antigen Group

### (1) F1 Antigen

The ‘F1’ antigen is the envelope or capsular protein of Y. pestis, and derives its name from ‘fraction 1’. It is also known as ‘caf1’, and is encoded on a plasmid. Cloning and sequencing of the F1 gene was reported in 1990 in reference 13 (GI:115437). In reference 11, the F1 antigen is referred to as ‘YPMT1.84’ (see GI:16082876). For reference purposes, the amino acid sequence of full-length F1 from the Y. pestis CO92 strain is given as SEQ ID NO:48 herein.

Preferred F1 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:48; and/or (b) that is a fragment of at least n consecutive amino acids of SEQ ID NO:48, wherein n is 7 or more (e.g. 8, 10, 12, 14, 16, 18, 20, 25, 30, 35, 40, 50, 60, 70, 80, 90, 100, 150, 200, 250 or more). These F1 proteins include variants of SEQ ID NO:48. Preferred fragments of (b) comprise an epitope from SEQ ID NO:48, and reference 13 suggests that the region located between amino acids 100 and 150 contains such epitopes. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:48. Other fragments omit one or more protein domains. For example, reference 97 discloses F1 proteins in which the 21-mer N-terminus signal peptide has been removed.

### (2) V Antigen

The V antigen is recognised as a major virulence factor of Y. pestis. In reference 11, the F1 antigen is referred to as ‘YPCD1.31c’, encoding the ‘antihost protein/regulator’ (see GI:5832451). It is also known as ‘lcrV’ for ‘low-calcium-response V’. For reference purposes, the amino acid sequence of full-length V antigen from the Y. pestis CO92 strain is given as SEQ ID NO:49 herein. Reference 14 reports V antigen sequences for 22 diverse strains of Y. pestis, with all but two being identical.

Preferred V antigens for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:49; and/or (b) that is a fragment of at least n consecutive amino acids of SEQ ID NO:49, wherein n is 7 or more (e.g. 8, 10, 12, 14, 16, 18, 20, 25, 30, 35, 40, 50, 60, 70, 80, 90, 100, 150, 200, 250 or more). These proteins include variants of SEQ ID NO:49. For example, GI:17380409 reports on sequence variants (K18N, K72R, I135V, C273S, and a mutant where ³²⁴SGK³²⁶ is replaced by R). Preferred fragments of (b) comprise an epitope from SEQ ID NO:49. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:49. Other fragments omit one or more protein domains.

### Sixth Antigen Group

### (1) YPO0457

The ‘YPO0457’ sequence was annotated in reference 11 as ‘hypothetical protein’ (see GI: 16120786). For reference purposes, the amino acid sequence of full-length YPO0457 as found in the Y. pestis CO92 strain is given as SEQ ID NO:61 herein. This protein is postulated herein to be a putative outer membrane protein.

Preferred YPO0457 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:61; and/or (b) that is a fragment of at least n consecutive amino acids of SEQ ID NO:61, wherein n is 7 or more (e.g. 8, 10, 12, 14, 16, 18, 20, 25, 30, 35, 40, 50, 60, 70, 80, 90, 100, 150, 200, 250 or more). These YPO0457 proteins include variants of SEQ ID NO:61. Preferred fragments of (b) comprise an epitope from SEQ ID NO:61. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:61. Other fragments omit one or more protein domains.

### (2) YPO0514

The ‘YPO0514’ sequence was annotated in reference 11 as ‘hypothetical protein’ (see GI: 16120845). For reference purposes, the amino acid sequence of full-length YPO0514 as found in the Y. pestis CO92 strain is given as SEQ ID NO:62 herein. However, it is postulated herein that YPO0514 forms part of a Type Three Secretion System (TTSS) and is an OmpA-family member protein.

Preferred YPO0514 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:62; and/or (b) that is a fragment of at least n consecutive amino acids of SEQ ID NO:62, wherein n is 7 or more (e.g. 8, 10, 12, 14, 16, 18, 20, 25, 30, 35, 40, 50, 60, 70, 80, 90, 100, 150, 200, 250 or more). These YPO0514 proteins include variants of SEQ ID NO:62. Preferred fragments of (b) comprise an epitope from SEQ ID NO:62. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:62. Other fragments omit one or more protein domains.

### (3) YPO0694

The ‘YPO0694’ sequence was annotated in reference 11 as ‘hypothetical protein’ (see GI: 16121015). For reference purposes, the amino acid sequence of full-length YPO0694 as found in the Y. pestis CO92 strain is given as SEQ ID NO:63 herein. This protein is postulated herein to be a putative membrane protein and furthermore, a fimbrial component.

Preferred YPO0694 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:63; and/or (b) that is a fragment of at least n consecutive amino acids of SEQ ID NO:63, wherein n is 7 or more (e.g. 8, 10, 12, 14, 16, 18, 20, 25, 30, 35, 40, 50, 60, 70, 80, 90, 100, 150, 200, 250 or more). These YPO0694 proteins include variants of SEQ ID NO:63. Preferred fragments of (b) comprise an epitope from SEQ ID NO:63. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:63. Other fragments omit one or more protein domains.

### (4) YPO0805

The ‘YPO0805’ sequence was annotated in reference 11 as ‘putative lipoprotein’ (see GI: 16121117). For reference purposes, the amino acid sequence of full-length YPO0805 as found in the Y. pestis CO92 strain is given as SEQ ID NO:64 herein. This protein is postulated herein to be a member of a virulence-associated secretion apparatus.

Preferred YPO0805 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:64; and/or (b) that is a fragment of at least n consecutive amino acids of SEQ ID NO:64, wherein n is 7 or more (e.g. 8, 10, 12, 14, 16, 18, 20, 25, 30, 35, 40, 50, 60, 70, 80, 90, 100, 150, 200, 250 or more). These YPO0805 proteins include variants of SEQ ID NO:64. Preferred fragments of (b) comprise an epitope from SEQ ID NO:64. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:64. Other fragments omit one or more protein domains.

### (5) YPO0982

The ‘YPO0982’ sequence was annotated in reference 11 as ‘putative lipoprotein’ (see GI: 16121286). For reference purposes, the amino acid sequence of full-length YPO0982 as found in the Y. pestis CO92 strain is given as SEQ ID NO:65 herein.

Preferred YPO0982 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:65; and/or (b) that is a fragment of at least n consecutive amino acids of SEQ ID NO:65, wherein n is 7 or more (e.g. 8, 10, 12, 14, 16, 18, 20, 25, 30, 35, 40, 50, 60, 70, 80, 90, 100, 150, 200, 250 or more). These YPO0982 proteins include variants of SEQ ID NO:65. Preferred fragments of (b) comprise an epitope from SEQ ID NO:65. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:65. Other fragments omit one or more protein domains.

### (6) YPO1354

The ‘YPO1354’ sequence was annotated in reference 11 as ‘putative lipoprotein’ (see GI: 16121634). For reference purposes, the amino acid sequence of full-length YPO1354 as found in the Y. pestis CO92 strain is given as SEQ ID NO:66 herein.

Preferred YPO1354 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:66; and/or (b) that is a fragment of at least n consecutive amino acids of SEQ ID NO:66, wherein n is 7 or more (e.g. 8, 10, 12, 14, 16, 18, 20, 25, 30, 35, 40, 50, 60, 70, 80, 90, 100, 150, 200, 250 or more). These YPO1354 proteins include variants of SEQ ID NO:66. Preferred fragments of (b) comprise an epitope from SEQ ID NO:66. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:66. Other fragments omit one or more protein domains.

### (7) YPO1408

The ‘YPO1408’ sequence was annotated in reference 11 as ‘hypothetical protein’ (see GI: 16121688). For reference purposes, the amino acid sequence of full-length YPO1408 as found in the Y. pestis CO92 strain is given as SEQ ID NO:67 herein. This protein is postulated herein to be a putative exported protein and a member of a type IV secretion system.

Preferred YPO1408 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:67; and/or (b) that is a fragment of at least n consecutive amino acids of SEQ ID NO:67, wherein n is 7 or more (e.g. 8, 10, 12, 14, 16, 18, 20, 25, 30, 35, 40, 50, 60, 70, 80, 90, 100, 150, 200, 250 or more). These YPO1408 proteins include variants of SEQ ID NO:67. Preferred fragments of (b) comprise an epitope from SEQ ID NO:67. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:67. Other fragments omit one or more protein domains.

### (8) YPO1792

The ‘YPO1792’ sequence was annotated in reference 11 as ‘flagellar protein FlhE precursor’ (see GI: 16122046). For reference purposes, the amino acid sequence of full-length YPO1792 as found in the Y. pestis CO92 strain is given as SEQ ID NO:68 herein.

Preferred YPO1792 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:68; and/or (b) that is a fragment of at least n consecutive amino acids of SEQ ID NO:68, wherein n is 7 or more (e.g. 8, 10, 12, 14, 16, 18, 20, 25, 30, 35, 40, 50, 60, 70, 80, 90, 100, 150, 200, 250 or more). These YPO1792 proteins include variants of SEQ ID NO:68. Preferred fragments of (b) comprise an epitope from SEQ ID NO:68. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:68. Other fragments omit one or more protein domains. A YPO1792 antigen has been shown to be an effective antigen for immunisation against lethal respiratory challenge with Y. pestis [15].

### (9) YPO2506

The ‘YPO2506’ sequence was annotated in reference 11 as ‘outer membrane protein X’ (see GI: 16122727). For reference purposes, the amino acid sequence of full-length YPO2506 as found in the Y. pestis CO92 strain is given as SEQ ID NO:69 herein.

Preferred YPO2506 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:69; and/or (b) that is a fragment of at least n consecutive amino acids of SEQ ID NO:69, wherein n is 7 or more (e.g. 8, 10, 12, 14, 16, 18, 20, 25, 30, 35, 40, 50, 60, 70, 80, 90, 100, 150, 200, 250 or more). These YPO2506 proteins include variants of SEQ ID NO:69. Preferred fragments of (b) comprise an epitope from SEQ ID NO:69. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:69. Other fragments omit one or more protein domains.

### (10) YPO2713

The ‘YPO2713’ sequence was annotated in reference 11 as ‘periplasmic negative regulator of sigmaE’ (see GI: 16122917). For reference purposes, the amino acid sequence of full-length YPO2713 as found in the Y. pestis CO92 strain is given as SEQ ID NO:70 herein.

Preferred YPO2713 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:70; and/or (b) that is a fragment of at least n consecutive amino acids of SEQ ID NO:70, wherein n is 7 or more (e.g. 8, 10, 12, 14, 16, 18, 20, 25, 30, 35, 40, 50, 60, 70, 80, 90, 100, 150, 200, 250 or more). These YPO2713 proteins include variants of SEQ ID NO:70. Preferred fragments of (b) comprise an epitope from SEQ ID NO:70. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:70. Other fragments omit one or more protein domains.

### (11) YPO2950

The ‘YPO2950’ sequence was annotated in reference 11 as ‘putative fimbrial protein’ (see GI: 16123133). For reference purposes, the amino acid sequence of full-length YPO2950 as found in the Y. pestis CO92 strain is given as SEQ ID NO:71 herein.

Preferred YPO2950 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:71; and/or (b) that is a fragment of at least n consecutive amino acids of SEQ ID NO:71, wherein n is 7 or more (e.g. 8, 10, 12, 14, 16, 18, 20, 25, 30, 35, 40, 50, 60, 70, 80, 90, 100, 150, 200, 250 or more). These YPO2950 proteins include variants of SEQ ID NO:71. Preferred fragments of (b) comprise an epitope from SEQ ID NO:71. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:71. Other fragments omit one or more protein domains.

### (12) YPO3026

The ‘YPO3026’ sequence was annotated in reference 11 as ‘putative lipoprotein’ (see GI: 16123203). For reference purposes, the amino acid sequence of full-length YPO3026 as found in the Y. pestis CO92 strain is given as SEQ ID NO:72 herein. This protein is postulated herein to be a pilin component.

Preferred YPO3026 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:72; and/or (b) that is a fragment of at least n consecutive amino acids of SEQ ID NO:72, wherein n is 7 or more (e.g. 8, 10, 12, 14, 16, 18, 20, 25, 30, 35, 40, 50, 60, 70, 80, 90, 100, 150, 200, 250 or more). These YPO3026 proteins include variants of SEQ ID NO:72. Preferred fragments of (b) comprise an epitope from SEQ ID NO:72. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:72. Other fragments omit one or more protein domains.

### (13) YPO3417

The ‘YPO3417’ sequence was annotated in reference 11 as ‘dihydrolipoamide dehydrogenase’ (see GI: 16123566). For reference purposes, the amino acid sequence of full-length YPO3417 as found in the Y. pestis CO92 strain is given as SEQ ID NO:73 herein.

Preferred YPO3417 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:73; and/or (b) that is a fragment of at least n consecutive amino acids of SEQ ID NO:73, wherein n is 7 or more (e.g. 8, 10, 12, 14, 16, 18, 20, 25, 30, 35, 40, 50, 60, 70, 80, 90, 100, 150, 200, 250 or more). These YPO3417 proteins include variants to of SEQ ID NO:73. Preferred fragments of (b) comprise an epitope from SEQ ID NO:73. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:73. Other fragments omit one or more protein domains.

### (14) YPO3551

The ‘YPO3551’ sequence was annotated in reference 11 as ‘hypothetical protein’ (see GI: 16123695). For reference purposes, the amino acid sequence of full-length YPO3551 as found in the Y. pestis CO92 strain is given as SEQ ID NO:74 herein. This protein is postulated herein to be a putative exported protein.

Preferred YPO3551 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:74; and/or (b) that is a fragment of at least n consecutive amino acids of SEQ ID NO:74, wherein n is 7 or more (e.g. 8, 10, 12, 14, 16, 18, 20, 25, 30, 35, 40, 50, 60, 70, 80, 90, 100, 150, 200, 250 or more). These YPO3551 proteins include variants of SEQ ID NO:74. Preferred fragments of (b) comprise an epitope from SEQ ID NO:74. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:74. Other fragments omit one or more protein domains.

### (15) YPO3646

The ‘YPO3646’ sequence was annotated in reference 11 as ‘outer membrane lipoprotein’ (see GI: 16123788). For reference purposes, the amino acid sequence of full-length YPO3646 as found in the Y. pestis CO92 strain is given as SEQ ID NO:75 herein. This protein is postulated herein to play a role in membrane integrity.

Preferred YPO3646 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:75; and/or (b) that is a fragment of at least n consecutive amino acids of SEQ ID NO:75, wherein n is 7 or more (e.g. 8, 10, 12, 14, 16, 18, 20, 25, 30, 35, 40, 50, 60, 70, 80, 90, 100, 150, 200, 250 or more). These YPO3646 proteins include variants of SEQ ID NO:75. Preferred fragments of (b) comprise an epitope from SEQ ID NO:75. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:75. Other fragments omit one or more protein domains.

### (16) YPO3982

The ‘YPO3982’ sequence was annotated in reference 11 as ‘hypothetical protein’ (see GI: 16124109). For reference purposes, the amino acid sequence of full-length YPO3982 as found in the Y. pestis CO92 strain is given as SEQ ID NO:76 herein.

Preferred YPO3982 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:76; and/or (b) that is a fragment of at least n consecutive amino acids of SEQ ID NO:76, wherein n is 7 or more (e.g. 8, 10, 12, 14, 16, 18, 20, 25, 30, 35, 40, 50, 60, 70, 80, 90, 100, 150, 200, 250 or more). These YPO3982 proteins include variants of SEQ ID NO:76. Preferred fragments of (b) comprise an epitope from SEQ ID NO:76. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:76. Other fragments omit one or more protein domains.

### (17) YPO0065

The ‘YPO0065’ sequence was annotated in reference 11 as ‘hypothetical protein’ (see GI: 16120416). For reference purposes, the amino acid sequence of full-length YPO0065 as found in the Y. pestis CO92 strain is given as SEQ ID NO:77 herein. This protein is postulated herein to be a putative membrane protein.

Preferred YPO0065 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:77; and/or (b) that is a fragment of at least n consecutive amino acids of SEQ ID NO:77, wherein n is 7 or more (e.g. 8, 10, 12, 14, 16, 18, 20, 25, 30, 35, 40, 50, 60, 70, 80, 90, 100, 150, 200, 250 or more). These YPO0065 proteins include variants of SEQ ID NO:77. Preferred fragments of (b) comprise an epitope from SEQ ID NO:77. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:77. Other fragments omit one or more protein domains.

### (18) YPO0499

The ‘YPO0499’ sequence was annotated in reference 11 as ‘hypothetical protein’ (see GI: 16120829). For reference purposes, the amino acid sequence of full-length YPO0499 as found in the Y. pestis CO92 strain is given as SEQ ID NO:78 herein. However, it is postulated herein that YPO0499 forms part of a Type Three Secretion System (TTSS).

Preferred YPO0499 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:78; and/or (b) that is a fragment of at least n consecutive amino acids of SEQ ID NO:78, wherein n is 7 or more (e.g. 8, 10, 12, 14, 16, 18, 20, 25, 30, 35, 40, 50, 60, 70, 80, 90, 100, 150, 200, 250 or more). These YPO0499 proteins include variants of SEQ ID NO:78. Preferred fragments of (b) comprise an epitope from SEQ ID NO:78. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:78. Other fragments omit one or more protein domains.

### (19) YPO0505

The ‘YPO0505’ sequence was annotated in reference 11 as ‘hypothetical protein’ (see GI: 16120835). For reference purposes, the amino acid sequence of full-length YPO0505 as found in the Y. pestis CO92 strain is given as SEQ ID NO:79 herein. However, it is postulated herein that YPO0505 forms part of a Type Three Secretion System (TTSS).

Preferred YPO0505 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:79; and/or (b) that is a fragment of at least n consecutive amino acids of SEQ ID NO:79, wherein n is 7 or more (e.g. 8, 10, 12, 14, 16, 18, 20, 25, 30, 35, 40, 50, 60, 70, 80, 90, 100, 150, 200, 250 or more). These YPO0505 proteins include variants of SEQ ID NO:79. Preferred fragments of (b) comprise an epitope from SEQ ID NO:79. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:79. Other fragments omit one or more protein domains.

### (20) YPO0500

The ‘YPO0500’ sequence was annotated in reference 11 as ‘hypothetical protein’ (see GI: 16120830). For reference purposes, the amino acid sequence of full-length YPO0500 as found in the Y. pestis CO92 strain is given as SEQ ID NO:80 herein. However, it is postulated herein that YPO0500 forms part of a Type Three Secretion System (TTSS).

Preferred YPO0500 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:80; and/or (b) that is a fragment of at least n consecutive amino acids of SEQ ID NO:80, wherein n is 7 or more (e.g. 8, 10, 12, 14, 16, 18, 20, 25, 30, 35, 40, 50, 60, 70, 80, 90, 100, 150, 200, 250 or more). These YPO0500 proteins include variants of SEQ ID NO:80. Preferred fragments of (b) comprise an epitope from SEQ ID NO:80. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:80. Other fragments omit one or more protein domains.

### (21) YPO0503

The ‘YPO0503’ sequence was annotated in reference 11 as ‘hypothetical protein’ (see GI: 16120833). For reference purposes, the amino acid sequence of full-length YPO0503 as found in the Y. pestis CO92 strain is given as SEQ ID NO:81 herein. However, it is postulated herein that YPO0503 forms part of a Type Three Secretion System (TTSS).

Preferred YPO0503 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:81; and/or (b) that is a fragment of at least n consecutive amino acids of SEQ ID NO:81, wherein n is 7 or more (e.g. 8, 10, 12, 14, 16, 18, 20, 25, 30, 35, 40, 50, 60, 70, 80, 90, 100, 150, 200, 250 or more). These YPO0503 proteins include variants of SEQ ID NO:81. Preferred fragments of (b) comprise an epitope from SEQ ID NO:81. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:81. Other fragments omit one or more protein domains.

### (22) YPO0506

The ‘YPO0506’ sequence was annotated in reference 11 as ‘putative Clp ATPase’ (see GI: 16120836). For reference purposes, the amino acid sequence of full-length YPO0506 as found in the Y. pestis CO92 strain is given as SEQ ID NO:82 herein. However, it is postulated herein that YPO0506 forms part of a Type Three Secretion System (TTSS).

Preferred YPO0506 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:82; and/or (b) that is a fragment of at least n consecutive amino acids of SEQ ID NO:82, wherein n is 7 or more (e.g. 8, 10, 12, 14, 16, 18, 20, 25, 30, 35, 40, 50, 60, 70, 80, 90, 100, 150, 200, 250 or more). These YPO0506 proteins include variants of SEQ ID NO:82. Preferred fragments of (b) comprise an epitope from SEQ ID NO:82. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:82. Other fragments omit one or more protein domains.

### (23) YPO0508

The ‘YPO0508’ sequence was annotated in reference 11 as ‘hypothetical protein’ (see GI: 16120838). For reference purposes, the amino acid sequence of full-length YPO0508 as found in the Y. pestis CO92 strain is given as SEQ ID NO:83 herein. However, it is postulated herein that YPO0508 forms part of a Type Three Secretion System (TTSS).

Preferred YPO0508 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:83; and/or (b) that is a fragment of at least n consecutive amino acids of SEQ ID NO:83, wherein n is 7 or more (e.g. 8, 10, 12, 14, 16, 18, 20, 25, 30, 35, 40, 50, 60, 70, 80, 90, 100, 150, 200, 250 or more). These YPO0508 proteins include variants of SEQ ID NO:83. Preferred fragments of (b) comprise an epitope from SEQ ID NO:83. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:83. Other fragments omit one or more protein domains.

### (24) YPO0509

The ‘YPO0509’ sequence was annotated in reference 11 as ‘hypothetical protein’ (see GI: 16120839). For reference purposes, the amino acid sequence of full-length YPO0509 as found in the Y. pestis CO92 strain is given as SEQ ID NO:84 herein. However, it is postulated herein that YPO0509 forms part of a Type Three Secretion System (TTSS).

Preferred YPO0509 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:84; and/or (b) that is a fragment of at least n consecutive amino acids of SEQ ID NO:84, wherein n is 7 or more (e.g. 8, 10, 12, 14, 16, 18, 20, 25, 30, 35, 40, 50, 60, 70, 80, 90, 100, 150, 200, 250 or more). These YPO0509 proteins include variants of SEQ ID NO:84. Preferred fragments of (b) comprise an epitope from SEQ ID NO:84. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:84. Other fragments omit one or more protein domains.

### (25) YPO3579

The ‘YPO3579’ sequence was annotated in reference 11 as ‘hypothetical protein’ (see GI: 16123723). For reference purposes, the amino acid sequence of full-length YPO3579 as found in the Y. pestis CO92 strain is given as SEQ ID NO:85 herein. This protein is postulated herein to be a putative exported protein.

Preferred YPO3579 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:85; and/or (b) that is a fragment of at least n consecutive amino acids of SEQ ID NO:85, wherein n is 7 or more (e.g. 8, 10, 12, 14, 16, 18, 20, 25, 30, 35, 40, 50, 60, 70, 80, 90, 100, 150, 200, 250 or more). These YPO3579 proteins include variants of SEQ ID NO:85. Preferred fragments of (b) comprise an epitope from SEQ ID NO:85. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:85. Other fragments omit one or more protein domains.

### (26) YPO4040

The ‘YPO4040’ sequence was annotated in reference 11 as ‘hypothetical protein’ (see GI: 16124160). For reference purposes, the amino acid sequence of full-length YPO4040 as found in the Y. pestis CO92 strain is given as SEQ ID NO:86 herein. This protein is postulated herein to be a putative exported protein and furthermore to be a fimbrial component.

Preferred YPO4040 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:86; and/or (b) that is a fragment of at least n consecutive amino acids of SEQ ID NO:86, wherein n is 7 or more (e.g. 8, 10, 12, 14, 16, 18, 20, 25, 30, 35, 40, 50, 60, 70, 80, 90, 100, 150, 200, 250 or more). These YPO4040 proteins include variants of SEQ ID NO:86. Preferred fragments of (b) comprise an epitope from SEQ ID NO:86. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:86. Other fragments omit one or more protein domains.

### Seventh Antigen Group

### (1) YPO0496

The ‘YPO0496’ sequence was annotated in reference 11 as ‘hypothetical protein’ (see GI: 16120826). For reference purposes, the amino acid sequence of full-length YPO0496 as found in the Y. pestis CO92 strain is given as SEQ ID NO: 87 herein.

Preferred YPO0496 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:87; and/or (b) that is a fragment of at least n consecutive amino acids of SEQ ID NO:87, wherein n is 7 or more (e.g. 8, 10, 12, 14, 16, 18, 20, 25, 30, 35, 40, 50, 60, 70, 80, 90, 100, 150, 200, 250 or more). These YPO0496 proteins include variants of SEQ ID NO:87. Preferred fragments of (b) comprise an epitope from SEQ ID NO:87. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:87. Other fragments omit one or more protein domains.

### (2) YPO1224

The ‘YPO1224’ sequence was annotated in reference 11 as ‘putative penicillin-bindin protein’ (see GI: 16121513). For reference purposes, the amino acid sequence of full-length YPO1224 as found in the Y. pestis CO92 strain is given as SEQ ID NO: 88 herein.

Preferred YPO1224 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:88; and/or (b) that is a fragment of at least n consecutive amino acids of SEQ ID NO:88, wherein n is 7 or more (e.g. 8, 10, 12, 14, 16, 18, 20, 25, 30, 35, 40, 50, 60, 70, 80, 90, 100, 150, 200, 250 or more). These YPO1224 proteins include variants of SEQ ID NO:88. Preferred fragments of (b) comprise an epitope from SEQ ID NO:88. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:88. Other fragments omit one or more protein domains.

### (3) YPO3553

The ‘YPO3553’ sequence was annotated in reference 11 as ‘enhancing lycopene biosynthesis protein 2’ (see GI: 16123697). For reference purposes, the amino acid sequence of full-length YPO3553 as found in the Y. pestis CO92 strain is given as SEQ ID NO: 89 herein.

Preferred YPO3553 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:89; and/or (b) that is a fragment of at least n consecutive amino acids of SEQ ID NO:89, wherein n is 7 or more (e.g. 8, 10, 12, 14, 16, 18, 20, 25, 30, 35, 40, 50, 60, 70, 80, 90, 100, 150, 200, 250 or more). These YPO3553 proteins include variants of SEQ ID NO:89. Preferred fragments of (b) comprise an epitope from SEQ ID NO:89. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:89. Other fragments omit one or more protein domains.

### (4) YPO3987

The ‘YPO3987’ sequence was annotated in reference 11 as ‘hypothetical protein’ (see GI: 16124114). For reference purposes, the amino acid sequence of full-length YPO3987 as found in the Y. pestis CO92 strain is given as SEQ ID NO: 90 herein. It has been suggested that this protein is an exported protein.

Preferred YPO3987 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:90; and/or (b) that is a fragment of at least n consecutive amino acids of SEQ ID NO:90, wherein n is 7 or more (e.g. 8, 10, 12, 14, 16, 18, 20, 25, 30, 35, 40, 50, 60, 70, 80, 90, 100, 150, 200, 250 or more). These YPO3987 proteins include variants of SEQ ID NO:90. Preferred fragments of (b) comprise an epitope from SEQ ID NO:90. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:90. Other fragments omit one or more protein domains.

### (5) YPO2190

The ‘YPO2190’ sequence was annotated in reference 11 as ‘attachment invasion locus protein precursor’ (see GI: 16122420). For reference purposes, the amino acid sequence of full-length YPO2190 as found in the Y. pestis CO92 strain is given as SEQ ID NO: 91 herein.

Preferred YPO2190 proteins for use with the invention comprise an amino acid sequence: (a) that has 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:91; and/or (b) that is a fragment of at least n consecutive amino acids of SEQ ID NO:91, wherein n is 7 or more (e.g. 8, 10, 12, 14, 16, 18, 20, 25, 30, 35, 40, 50, 60, 70, 80, 90, 100, 150, 200, 250 or more). These YPO2190 proteins include variants of SEQ ID NO:91. Preferred fragments of (b) comprise an epitope from SEQ ID NO:91. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO:91. Other fragments omit one or more protein domains.

### Type Three Secretion System

The Y. pestis proteins YPO0499, YPO0500, YPO0501, YPO0502, YPO0503, YPO0504, YPO0505, YPO0506, YPO0507, YPO0508, YPO0509, YPO0510, YPO0511, YPO0512, YPO0513, YPO0514, YPO0515 and YPO0516 are postulated herein to form part of a Type Three Secretion System (TTSS). Analysis reveals sequence similarity between these proteins and those of the Icm/Dot secretion system, also known as the IcmF-associated homologous protein (IAHP) gene cluster of Legionella pneumophila [16-20]. Furthermore, YPO0499-YPO0506 have sequence similarity with proteins of the EVP cluster, which forms a secretion system in Edwardsiella tarda [21]. A further Type Three Secretion System has recently been described in Vibrio cholerae. Elements of this Vibrio system share identity with proteins of the system share identity with proteins of the Y. pestis cluster YPO0499-YPO0516.

Of these proteins, YPO0499, YPO0500, YPO0501, YPO0502, YPO0503, YPO0505, YPO0506, YPO0508, YPO0509, YPO0512 and YPO0514 are considered to be surface exposed and therefore useful as immunising antigens.

Thus, particularly preferred compositions of the invention comprise one or more (i.e. 1, 2, 3, 4, 5, 6, 7, 8, 9, 10 or all 11) of YPO0499, YPO0500, YPO0501, YPO0502, YPO0503, YPO0505, YPO0506, YPO0508, YPO0509, YPO0512 and/or YPO0514. YPPO499 and YPPO502 can be used for immunisation separately or in combination, optionally with one or more further TTSS protein(s).

As noted above, a particularly preferred composition comprises YPO0499, YPO0502 and YPO0505.

### Fusion and Hybrid Polypeptides

The Y. pestis antigens used in the invention may be present in the composition as individual separate polypeptides. Where more than one antigen is used, however, they do not have to be present as separate polypeptides. Instead, at least two (e.g. 2, 3, 4, 5, or more) antigens can be expressed as a single polypeptide chain (a ‘hybrid’ polypeptide). Hybrid polypeptides offer two main advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful. The F1 and V antigens, for instance, can be expressed as a hybrid [22].

The hybrid polypeptide may comprise two or more polypeptide sequences from the first antigen group. The hybrid polypeptide may comprise one or more polypeptide sequences from the first antigen group and one or more polypeptide sequences from the second antigen group. The hybrid polypeptide may comprise one or more polypeptide sequences from the first antigen group and one or more polypeptide sequences from the third antigen group. The hybrid polypeptide may comprise one or more polypeptide sequences from the second antigen group and one or more polypeptide sequences from the third antigen group. The hybrid polypeptide may comprise one or more polypeptide sequences from the first, second and/or third antigen group and one or more polypeptide sequences from the fourth antigen group. The hybrid polypeptide may comprise one or more polypeptide sequences from the first, second and/or third antigen group and one or more polypeptide sequences from the fifth antigen group. The hybrid polypeptide may comprise one or more polypeptide sequences from the first, second and/or third antigen group and one or more polypeptide sequences from the sixth antigen group. The hybrid polypeptide may comprise one or more polypeptide sequences from the first, second and/or third antigen group and one or more polypeptide sequences from the seventh antigen group.

Hybrids for use in the present invention may also comprise combinations of antigens selected from the second, third, fourth, fifth, sixth and seventh antigen groups.

Hybrids consisting of amino acid sequences from two, three, four, five, six, seven, eight, nine, or ten Y. pestis antigens are preferred. In particular, hybrids consisting of amino acid sequences from two, three, four, or five Y. pestis antigens are preferred. Particularly preferred are hybrids consisting of amino acid sequences from two or three Y. pestis antigens.

A preferred hybrid protein according to the invention comprises two or more (i.e. 2, 3, 4, 5, 6, 7, 8, 9, 10, 11, 12 or all 13) of a YPO0499 antigen, a YPO0502 antigen, a YPO0505 antigen, a YPO0809 antigen, a YPO1070 antigen, a YPO1123 antigen, a YPO1604 antigen, a YPO2881 antigen, a YPO3489 antigen, a YPO1411 antigen, a YPO3935 antigen, a YPO3982 antigen and a YPO4003 antigen.

Particularly preferred hybrid proteins according to the invention comprise (i) a YPO0499 antigen, a YPO3489 antigen, a YPO4003 antigen and a YPO1604 antigen, (ii) a YPO0499 antigen, a YPO0502 antigen and a YPO0505 antigen, (iii) a YPO1070 antigen, a YPO1123 antigen, a YPO2881 antigen and a YPO0809 antigen, or (iv) a YPO1411 antigen, a YPO3935 antigen and a YPO3982 antigen.

Different hybrid polypeptides may be mixed together in a single formulation. Within such combinations, a Y. pestis antigen may be present in more than one hybrid polypeptide and/or as a non-hybrid polypeptide. It is preferred, however, that an antigen is present either as a hybrid or as a non-hybrid, but not as both.

Hybrid polypeptides can be represented by the formula NH₂-A-{-X-L-}ₙ-B—COOH, wherein: X is an amino acid sequence of a Y. pestis antigen, as described above; L is an optional linker amino acid sequence; A is an optional N-terminal amino acid sequence; B is an optional C-terminal amino acid sequence; n is an integer of 2 or more (e.g. 2, 3, 4, 5, 6, 7, 8, 9, 10, 11, 12, 13, 14 or 15). Most preferably, n is 2 or 3.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {-X-L-}, linker amino acid sequence -L- may be present or absent. For instance, when n=2 the hybrid may be NH₂—X₁-L₁-X₂-L₂-COOH, NH₂—X₁-X₂—COOH, NH₂—X₁-L₁-X₂—COOH, NH₂—X₁-X₂-L₂-COOH, etc. Linker amino acid sequence(s)-L- will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Gly where n=2, 3, 4, 5, 6, 7, 8, 9, 10 or more), and histidine tags (i.e. Hisₙ where n=3, 4, 5, 6, 7, 8, 9, or more). Other suitable linker amino acid sequences will be apparent to those skilled in the art. A useful linker is GSGGGG (SEQ ID NO:60), with the Gly-Ser dipeptide being formed from a BamHI restriction site, thus aiding cloning and manipulation, and the (Gly)₄ tetrapeptide being a typical poly-glycine linker.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art.

Preferred fusion protein compositions of the invention comprise one or more (i.e. 1, 2, 3, 4, 5, 6, 7, 8, 9, 10 or more) of 0809_GST, 0809_His, 0499_GST, 0499_His, 1070_GST, 1070_His, 3489_GST, 3489_His, 1354_GST, 1354_His, 3631_GST, 3631_His, 1604_GST, 1604_His, 4003_GST, 4003_His, 0500_His, 0501_His, 0502_His, 0502_GST, 0503_His, 0503 GST, 0505_His, 0505GST, 0506_His, 0508_GST and/or 0509_GST. According to this nomenclature, each antigen may have a N-terminal GST tag or a C-terminal his tag. Therefore, for example, 3489_His is YPO3489 with a C-terminal his tag and 0809_GST is YPO0809 with a N-terminal GST tag.

Particularly preferred combinations comprise (1) 0809_GST and 0499_GST, (2) 1070_GST and 3489_His, (3) 1354_His and 3631_His, and/or (4) 1604_His and 4003_His. Such preferred combinations may be found in an immunogenic composition further comprising alum and/or CpG.

The invention also provides nucleic acid encoding hybrid polypeptides of the invention. The term “nucleic acid” includes DNA and RNA, and also their analogues, such as those containing modified backbones (e.g. phosphorothioates, etc.), and also peptide nucleic acids (PNA), etc.

Polypeptides Used with the Invention

Polypeptides used with the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.). F1, for instance, is known to exist in various forms, including a multimeric glycoprotein form. Lipoproteins are particularly preferred for use as immunogens.

Polypeptides used with the invention can be prepared by various means (e.g. recombinant expression, purification from cell culture, chemical synthesis, etc.). Recombinantly-expressed proteins are preferred, particularly for hybrid polypeptides.

Polypeptides used with the invention are preferably provided in purified or substantially purified form i.e. substantially free from other polypeptides (e.g. free from naturally-occurring polypeptides), particularly from other Yersinia or host cell polypeptides, and are generally at least about 50% pure (by weight), and usually at least about 90% pure i.e. less than about 50%, and more preferably less than about 10% (e.g. 5%) of a composition is made up of other expressed polypeptides. Thus the antigens in the compositions are separated from the whole organism with which the molecule is expressed.

Polypeptides used with the invention are preferably Y. pestis polypeptides.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included are, for example, polypeptides containing one or more analogs of an amino acid (including, for example, unnatural amino acids, etc.), as well as other modifications known in the art. Polypeptides can occur as single chains or associated chains.

The invention provides polypeptides comprising a sequence —P-Q- or -Q-P—, wherein: —P— is an amino acid sequence as defined above and -Q- is not a sequence as defined above i.e. the invention provides fusion proteins. Fusion proteins of F1 are known, for instance, from reference 97, where a heterologous anchor domain is attached to allow cell-surface display of a protein that would normally be secreted. Where the N-terminus codon of —P— is not ATG, but this codon is not present at the N-terminus of a polypeptide, it will be translated as the standard amino acid for that codon rather than as a Met. Where this codon is at the N-terminus of a polypeptide, however, it will be translated as Met. Polypeptides used with the invention may be prepared as a GST-fusion protein and/or a His-tagged fusion protein.

The invention also provides a process for producing a polypeptide of the invention, comprising the step of culturing a host cell transformed with nucleic acid of the invention under conditions which induce polypeptide expression.

The invention provides a process for producing a polypeptide of the invention, comprising the step of synthesising at least part of the polypeptide by chemical means.

### Strains

Polypeptides of the invention may comprise an amino acid sequence found in a Y. pestis of biovar Antigua, Mediaevalis, Orientalis and/or Microtus, with biovar orientalis being preferred [23].

Polypeptides of the invention may comprise an amino acid sequence found in a Y. pestis of ribotypes A, B, C, Q, R, and/or T.

Preferred polypeptides of the invention comprise an amino acid sequence found in Y. pestis strains C092 [11], KIM [24], 91001 [25], 685, etc., including the strains listed in references 23 and 94. The sequence may also be found in other Yersinia species, such as a Y. pseudotuberculosis (full genome sequence available as GI: 51587641 [26]) or a Y. enterocolitica.

Where hybrid polypeptides are used, the individual antigens within the hybrid (i.e. individual —X-moieties) may be from one or more strains. Where n=2, for instance, X₂ may be from the same strain as X₁ or from a different strain. Where n=3, the strains might be (i) X₁=X₂=X₃ (ii) X₁=X₂≠X₃ (iii) X₁≠X₂=X₃ (iv) X₁≠X₂≠X₃ or (v) X₁=X₃≠X₂, etc.

### Heterologous Hosts

Whilst expression of the polypeptides of the invention may take place in Yersinia, the invention preferably utilises a heterologous host. The heterologous host may be prokaryotic (e.g. a bacterium) or eukaryotic. It is preferably E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

### Immunogenic Compositions and Medicaments

Compositions of the invention are preferably immunogenic compositions, such as vaccine compositions. The pH of the composition is preferably between 6 and 8, preferably about 7. The pH may be maintained by the use of a buffer. A phosphate buffer is typical. The composition may be sterile and/or pyrogen-free. The composition may be gluten-free. The composition may be substantially free from formaldehyde, phenol, beef-heart extract, yeast extract, and/or agar. The composition may be free from Y. pestis DNA. The composition may be isotonic with respect to humans.

Vaccines according to the invention may either be prophylactic (i.e. to prevent infection) or therapeutic (i.e. to treat infection), but will typically be prophylactic. Accordingly, the invention includes a method for the therapeutic or prophylactic treatment of Y. pestis infection in an animal susceptible to Yersinia infection comprising administering to said animal a therapeutic or prophylactic amount of the immunogenic compositions of the invention.

Compositions may include a preservative, particularly if packaged in a multiple dose format.

Compositions may comprise detergent e.g. a Tween (polysorbate), such as Tween 80. Detergents are generally present at low levels e.g. <0.01%.

Compositions may include sodium salts (e.g. sodium chloride) to give tonicity. A concentration of 10±2 mg/ml NaCl is typical.

Compositions may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material.

The immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below.

Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 27], or mixtures of different mineral compounds, with the compounds taking any suitable form (e.g. gel, crystalline, amorphous, etc.), and with adsorption being preferred. The mineral containing compositions may also be formulated as a particle of metal salt [28].

Aluminium phosphates are particularly preferred, particularly in compositions which include a H. influenzae saccharide antigen, and a typical adjuvant is amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose. Where there is more than one conjugate in a composition, not all conjugates need to be adsorbed.

### B. Oil Emulsions

Oil emulsion compositions suitable for use as adjuvants in the invention include squalene-water emulsions, such as MF59 [Chapter 10 of ref. 27; see also ref. 29] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

### C. Saponin Formulations [Chapter 22 of Ref 27]

Saponin formulations may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 30. Saponin formulations may also comprise a sterol, such as cholesterol [31].

Combinations of saponins and cholesterols can be used to form unique particles called immunostimulating complexs (ISCOMs) [chapter 23 of ref. 27]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 31-33. Optionally, the ISCOMS may be devoid of additional detergent [34].

A review of the development of saponin based adjuvants can be found in refs. 35 & 36.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 37-42. Virosomes are discussed further in, for example, ref. 43

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 44. Such “small particles” of 3dMPL are small enough to be sterile filtered through a 0.22 μm membrane [44]. Other non-toxic LPS derivatives include monophosphoryl lipid A mimics, such as aminoalkyl glucosaminide phosphate derivatives e.g. RC-529 [45,46].

Lipid A derivatives include derivatives of lipid A from Escherichia coli such as OM-174. OM-174 is described for example in refs. 47 & 48.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 49, 50 and 51 disclose possible analog substitutions e.g. replacement of guanosine with 2′-deoxy-7-deazaguanosine. The adjuvant effect of CpG oligonucleotides is further discussed in refs. 52-57.

The CpG sequence may be directed to TLR9, such as the motif GTCGTT or TTCGTT [58]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 59-61. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 58 & 62-64.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 65 and as parenteral adjuvants in ref. 66. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivaties thereof, particularly LT-K63 and LT-R72, as adjuvants can be found in refs. 67-74. Numerical reference for amino acid substitutions is preferably based on the alignments of the A and B subunits of ADP-ribosylating toxins set forth in ref. 75, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [76], etc.) [77], interferons (e.g. interferon-γ), macrophage colony stimulating factor, and tumor necrosis factor. A preferred immunomodulator is IL-12.

### G. Bioadhesives and Mucoadhesives

Bioadhesives and mucoadhesives may also be used as adjuvants in the invention. Suitable bioadhesives include esterified hyaluronic acid microspheres [78] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [79].

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of Ref 27)

Examples of liposome formulations suitable for use as adjuvants are described in refs. 80-82.

### J. Polyoxyethylene Ether and Polyoxyethylene Ester Formulations

Adjuvants suitable for use in the invention include polyoxyethylene ethers and polyoxyethylene esters [83]. Such formulations further include polyoxyethylene sorbitan ester surfactants in combination with an octoxynol [84] as well as polyoxyethylene alkyl ethers or ester surfactants in combination with at least one additional non-ionic surfactant such as an octoxynol [85]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Polyphosphazene (PCPP)

PCPP formulations are described, for example, in refs. 86 and 87.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e.g. “Resiquimod 3M”), described further in refs. 88 and 89.

The invention may also comprise combinations of aspects of one or more of the adjuvants identified above. For example, the following adjuvant compositions may be used in the invention: (1) a saponin and an oil-in-water emulsion [90]; (2) a saponin (e.g. QS21)+a non-toxic LPS derivative (e.g. 3dMPL) [91]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [92]; (5) combinations of 3dMPL with, for example, QS21 and/or oil-in-water emulsions [93]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 27.

The use of an aluminium hydroxide and/or aluminium phosphate adjuvant is particularly preferred, and antigens are generally adsorbed to these salts. Calcium phosphate is another preferred adjuvant. Other preferred adjuvant combinations include combinations of Th1 and Th2 adjuvants such as CpG & alum or resiquimod & alum.

Use of the combination of a mineral salt, such as an aluminium salt, and an oligonucleotide containing a CpG motif provide for an enhanced immune response. The invention therefore provides a composition comprising an oligonucleotide containing a CpG motif, a mineral salt such as an aluminium salt, and one or more Y. pestis antigens as defined above. The invention also provides a composition comprising an ADP ribosylating toxin (such as a detoxified ADP ribosylating toxin), an oligonucleotide containing a CpG motif, and one or more Y. pestis antigens as defined above.

The compositions of the invention will preferably elicit both a cell mediated immune response as well as a humoral immune response in order to effectively address a Yersinia intracellular infection. This immune response will preferably induce long lasting (e.g. neutralising) antibodies and a cell mediated immunity that can quickly respond upon exposure to Yersinia.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

Preferably, the invention includes a composition comprising a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

### Methods of Treatment and Medical Uses

The invention provides a combination comprising two or more (i.e. 2, 3, 4, 5, 6, 7, 8, 9, 10, 11, 12 or all 13) of a YPO0499 antigen, a YPO0502 antigen, a YPO0505 antigen, a YPO0809 antigen, a YPO1070 antigen, a YPO1123 antigen, a YPO1604 antigen, a YPO2881 antigen, a YPO3489 antigen, a YPO1411 antigen, a YPO3935 antigen, a YPO3982 antigen and a YPO4003 antigen for use (i) as an immunogen, (ii) in therapy, and/or (iii) in the manufacture of a medicament for raising an immune response in a mammal.

The invention also provides the use of a combination comprising two or more (i.e. 2, 3, 4, 5, 6, 7, 8, 9, 10, 11, 12 or all 13) of a YPO0499 antigen, a YPO0502 antigen, a YPO0505 antigen, a YPO0809 antigen, a YPO1070 antigen, a YPO1123 antigen, a YPO1604 antigen, a YPO2881 antigen, a YPO3489 antigen, a YPO1411 antigen, a YPO3935 antigen, a YPO3982 antigen and a YPO4003 antigen in the manufacture of a medicament for raising an immune response in a mammal.

The invention provides a combination comprising a YPO4003 antigen, a YPO1604 antigen, a YPO3489 antigen and a YPO0499 antigen for use (i) as an immunogen, (ii) in therapy, and/or (iii) in the manufacture of a medicament for raising an immune response in a mammal.

The invention also provides the use of a combination comprising a YPO4003 antigen, a YPO1604 antigen, a YPO3489 antigen and a YPO0499 antigen in the manufacture of a medicament for raising an immune response in a mammal.

The invention provides a combination comprising a YPO0499 antigen, a YPO0502 antigen and a YPO0505 antigen for use (i) as an immunogen, (ii) in therapy, and/or (iii) in the manufacture of a medicament for raising an immune response in a mammal.

The invention also provides the use of a combination comprising a YPO0499 antigen, a YPO0502 antigen and a YPO0505 antigen in the manufacture of a medicament for raising an immune response in a mammal.

The invention provides a combination comprising a YPO1070 antigen, a YPO1123 antigen, a YPO2881 antigen and a YPO0809 antigen for use (i) as an immunogen, (ii) in therapy, and/or (iii) in the manufacture of a medicament for raising an immune response in a mammal.

The invention also provides the use of a combination comprising a YPO1070 antigen, a YPO1123 antigen, a YPO2881 antigen and a YPO0809 antigen in the manufacture of a medicament for raising an immune response in a mammal.

The invention provides a combination comprising a YPO1411 antigen, a YPO3935 antigen and a YPO3982 antigen for use (i) as an immunogen, (ii) in therapy, and/or (iii) in the manufacture of a medicament for raising an immune response in a mammal.

The invention also provides the use of a combination comprising a YPO1411 antigen, a YPO3935 antigen and a YPO3982 antigen in the manufacture of a medicament for raising an immune response in a mammal.

The invention provides one or more of (1) a YPO0512 antigen; (2) a YPO0563 antigen; (3) a YPO3489 antigen; (4) a YPO4003 antigen; (5) a YPO1604 antigen; (6) a YPO3061 antigen; (7) a YPO3559 antigen; (8) a YPO3382 antigen; (9) a YPO0860 antigen; (10) a YPO0086 antigen; (11) a YPO3631 antigen; (12) a YPO2881 antigen; (13) a YPO3343 antigen; (14) a YPO3361 antigen; (15) a YPO3430 antigen; (16) a YPO1411 antigen; (17) a YPO3935 antigen; (18) a YPO0809 antigen; (19) a YPO1123 antigen; (20) a YPO3065 antigen; and/or (21) a YPO1070 antigen, for use (i) as an immunogen, (ii) in therapy, and/or (iii) in the manufacture of a medicament for raising an immune response in a mammal.

The invention also provides the use of one or more of (1) a YPO0512 antigen; (2) a YPO0563 antigen; (3) a YPO3489 antigen; (4) a YPO4003 antigen; (5) a YPO1604 antigen; (6) a YPO3061 antigen; (7) a YPO3559 antigen; (8) a YPO3382 antigen; (9) a YPO0860 antigen; (10) a YPO0086 antigen; (11) a YPO3631 antigen; (12) a YPO2881 antigen; (13) a YPO3343 antigen; (14) a YPO3361 antigen; (15) a YPO3430 antigen; (16) a YPO1411 antigen; (17) a YPO3935 antigen; (18) a YPO0809 antigen; (19) a YPO1123 antigen; (20) a YPO3065 antigen; and/or (21) a YPO1070 antigen, in the manufacture of a medicament for raising an immune response in a mammal.

The invention provides one or more of (1) a YPO0102 antigen; (2) a YPO0570 antigen; (3) a YPO1053 antigen; (4) a YPO1435 antigen; (5) a YPO2674 antigen; (6) a YPO2292 antigen; (7) a YPO3050 antigen; (8) a YPO2615 antigen; (9) a YPO1507 antigen; (10) a YPO4111 antigen; (11) a YPO0015 antigen; (12) a YPO0195 antigen; (13) a YPO2342 antigen; (14) a YPO0501 antigen; (15) a YPO0502 antigen; (16) a YPO0819 antigen; (17) a YPO3644 antigen; (18) a YPO1746 antigen; (19) a YPO0351 antigen; (20) a YPO0468 antigen; (21) a YPO0203 antigen; (22) a YPO0216 antigen; (23) a YPO3536 antigen; (24) a YPO0233 antigen; (25) a YPO0067 antigen; (26) a YPO3643 antigen; (27) a YPO3375 antigen; (28) a YPO0494 antigen; (29) a YPO1052 antigen; (30) a YPO1906 antigen; (31) a YPO0663 antigen; (32) a YPO1222 antigen; (33) a YPO2905 antigen; (34) a YPO4070 antigen; (35) a YPPCP1.07 antigen; and/or (36) a YPMT1.42 antigen, for use (i) as an immunogen, (ii) in therapy, and/or (iii) in the manufacture of a medicament for raising an immune response in a mammal.

The invention also provides the use of one or more of (1) a YPO0102 antigen; (2) a YPO0570 antigen; (3) a YPO1053 antigen; (4) a YPO1435 antigen; (5) a YPO2674 antigen; (6) a YPO2292 antigen; (7) a YPO3050 antigen; (8) a YPO2615 antigen; (9) a YPO1507 antigen; (10) a YPO4111 antigen; (11) a YPO0015 antigen; (12) a YPO0195 antigen; (13) a YPO2342 antigen; (14) a YPO0501 antigen; (15) a YPO0502 antigen; (16) a YPO0819 antigen; (17) a YPO3644 antigen; (18) a YPO1746 antigen; (19) a YPO0351 antigen; (20) a YPO0468 antigen; (21) a YPO0203 antigen; (22) a YPO0216 antigen; (23) a YPO3536 antigen; (24) a YPO0233 antigen; (25) a YPO0067 antigen; (26) a YPO3643 antigen; (27) a YPO3375 antigen; (28) a YPO0494 antigen; (29) a YPO1052 antigen; (30) a YPO1906 antigen; (31) a YPO0663 antigen; (32) a YPO1222 antigen; (33) a YPO2905 antigen; (34) a YPO4070 antigen; (35) a YPPCP1.07 antigen; and/or (36) a YPMT1.42 antigen, in the manufacture of a medicament for raising an immune response in a mammal.

The invention provides one or more of (1) a YPO0457 antigen; (2) a YPO0514 antigen; (3) a YPO0694 antigen; (4) a YPO0805 antigen; (5) a YPO0982 antigen; (6) a YPO1354 antigen; (7) a YPO1408 antigen; (8) a YPO1792 antigen; (9) a YPO2506 antigen; (10) a YPO2713 antigen; (11) a YPO2950 antigen; (12) a YPO3026 antigen; (13) a YPO3417 antigen; (14) a YPO3551 antigen; (15) a YPO3646 antigen; (16) a YPO3982 antigen; (17) a YPO0065 antigen; (18) a YPO0499 antigen; (19) a YPO0505 antigen, (20) a YPO0500 antigen; (21) a YPO0503 antigen; (22) a YPO0506 antigen; (23) a YPO0508 antigen; (24) a YPO0509 antigen; (25) a YPO3579 antigen and/or (26) a YPO4040 antigen, for use (i) as an immunogen, (ii) in therapy, and/or (iii) in the manufacture of a medicament for raising an immune response in a mammal.

The invention provides the use of one or more of (1) a YPO0457 antigen; (2) a YPO0514 antigen; (3) a YPO0694 antigen; (4) a YPO0805 antigen; (5) a YPO0982 antigen; (6) a YPO1354 antigen; (7) a YPO1408 antigen; (8) a YPO1792 antigen; (9) a YPO2506 antigen; (10) a YPO2713 antigen; (11) a YPO2950 antigen; (12) a YPO3026 antigen; (13) a YPO3417 antigen; (14) a YPO3551 antigen; (15) a YPO3646 antigen; (16) a YPO3982 antigen; (17) a YPO0065 antigen; (18) a YPO0499 antigen; (19) a YPO0505 antigen, (20) a YPO0500 antigen; (21) a YPO0503 antigen; (22) a YPO0506 antigen; (23) a YPO0508 antigen; (24) a YPO0509 antigen; (25) a YPO3579 antigen and/or (26) a YPO4040 antigen in the manufacture of a medicament for raising an immune response in a mammal.

The invention provides one or more of (1) a YPO0496 antigen; (2) a YPO1224 antigen; (3) a YPO3553 antigen; (4) a YPO3987 antigen and/or (5) a YPO2190 antigen, for use (i) as an immunogen, (ii) in therapy, and/or (iii) in the manufacture of a medicament for raising an immune response in a mammal.

The invention provides the use of one or more of (1) a YPO0496 antigen; (2) a YPO1224 antigen; (3) a YPO3553 antigen; (4) a YPO3987 antigen and/or (5) a YPO2190 antigen in the manufacture of a medicament for raising an immune response in a mammal.

These medicaments are preferably vaccines.

The invention also provides a method for raising an immune response in a mammal comprising the step of administering an effective amount of a composition of the invention. The immune response is preferably protective and preferably involves antibodies and/or cell-mediated immunity. The method may raise a booster response.

By raising an immune response in the mammal by these uses and methods, the mammal can be protected against Y. pestis infection. More particularly, the mammal may be protected against a plague, including bubonic plague, septicemic plague and/or pneumonic plague. Other related diseases include cellulocutaneous plague and plague meningitis. The medicament is preferably for protecting a mammal against pneumonic plague.

Compositions of the invention can preferably protect against Y. pestis ribotypes [94,95] including one or more of A, B, C, Q, R, and/or T.

Compositions of the invention can preferably protect against Y. pestis biovars including one or more of antiqua, mediaevalis, orientalis and/or microtus [96].

The invention also provides a kit comprising a first component and a second component wherein neither the first component nor the second component is a composition of the invention as described above, but wherein the first component and the second component can be combined to provide a composition of the invention as described above. The kit may further include a third component comprising one or more of the following: instructions, syringe or other delivery device, adjuvant, or pharmaceutically acceptable formulating solution.

The invention also provides a delivery device pre-filled with an immunogenic composition of the invention.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

One way of checking efficacy of therapeutic treatment involves monitoring Y. pestis infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the Y. pestis antigens in the compositions of the invention after administration of the composition. Typically, serum Yersinia specific antibody responses are determined post-immunisation but pre-challenge whereas mucosal Yersinia specific antibody body responses are determined post-immunisation and post-challenge. The protective effect of a composition can be tested in standard animal models, including the murine aerosol challenge model of reference 8.

Another way of assessing the immunogenicity of the compositions of the present invention is to express the proteins recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The vaccine compositions of the present invention can be evaluated in in vitro and in vivo animal models prior to host, e.g., human, administration. For example, in vitro neutralization is suitable for testing vaccine compositions directed toward Y. pestis.

The efficacy of vaccine compositions can also be determined in vivo by challenging animal models of Y. pestis infection, e.g., guinea pigs or mice, with the vaccine compositions. For example, reference 97 describes the immunisation of mice against Y. pestis and then challenging with F1 antigen. The administered compositions may or may not be derived from the same strains as the challenge strains. Preferably the compositions are derived from the same strains as the challenge strains. In vivo efficacy models include but are not limited to: (i) murine infection models using Y. pestis strains that are infectious to humans; (ii) murine disease models which use mouse-adapted Y. pestis strains, such as strains which are particularly virulent in mice; and (iii) primate models using human strains.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal (see e.g. reference 98) or transcutaneous (see e.g. references 99 and 100), intranasal (see e.g. reference 101), ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage treatment can be a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc.

Yersinia infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

### Further Components of the Composition

Yersinia antigens of the invention can be combined with pharmaceutically acceptable carriers. Such carriers include any carrier that does not itself induce the production of antibodies harmful to the individual receiving the composition. Such carriers are well known to those of ordinary skill in the art. The compositions may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in reference 102.

The invention further provides a method for preparing a pharmaceutical product, comprising the steps of: (a) preparing Yersinia antigens as described above; (b) mixing the antigens with one or more pharmaceutically acceptable carriers; and (c) packaging the antigen/carrier mixture into a container, such as a vial or a syringe, to give a pharmaceutical product. Insertion into a syringe may be performed in a factory or in a surgery.

The compositions can also include non-Yersinia immunogens. Thus the compositions may include one or more of: an immunogen from Bacillus anthracis for protecting against anthrax infection (e.g. a PA antigen [103], a spore antigen, etc.); an immunogen from a bacterium in the Francisella genus, such as F. tularensis for protecting against tularemia; an immunogen from a bacterium in the Pasteurella genus; an immunogen from a bacterium in the Brucella genus for protecting against brucellosis, such as B. abortus, B. melitensis, or B. suis; an immunogen from a bacterium in the Burkholderia genus, such as B. mallei for protecting against glanders or B. pseudomallei for protecting against melioidosis; an immunogen from a bacterium in the Chlamydia genus, such as Chlamydia psittaci for protecting against psittacosis; an immunogen from a bacterium in the Clostridium genus, such as C. botulinum for protecting against botulism or C. perfringens for protecting against Epsilon toxin); an immunogen from a bacterium in the Francisella genus, such as F. tularensis for protecting against tularemia; an immunogen from a Vibrio cholerae bacterium for protecting against cholera; an immunogen from a Coxiella burnetii bacterium for protecting against Q fever; an immunogen from an Ebola virus and/or a Marburg virus and/or a Lassa virus and/or a Machupo virus, for protecting against hemorrhagic fever; an immunogen from a bacterium in the Rickettsia genus, such as R. prowazekii bacterium for protecting against typhus fever, or from R. rickettsii; an immunogen from a fungus in the Coccidioides genus, such as C. immitis or C. posadasii; etc.

### Nucleic Acid Immunisation

The immunogenic compositions described above include polypeptide antigens from Y. pestis. In all cases, however, the polypeptide antigens can be replaced by nucleic acids (typically DNA) encoding those polypeptides, to give compositions, methods and uses based on nucleic acid immunisation. Nucleic acid immunisation is now a developed field (e.g. see references 97 and 104 to 111 etc.), and has been applied to Y. pestis vaccines [112-117].

The nucleic acid encoding the immunogen is expressed in vivo after delivery to a patient and the expressed immunogen then stimulates the immune system. The active ingredient will typically take the form of a nucleic acid vector comprising: (i) a promoter; (ii) a sequence encoding the immunogen, operably linked to the promoter; and optionally (iii) a selectable marker. Preferred vectors may further comprise (iv) an origin of replication; and (v) a transcription terminator downstream of and operably linked to (ii). In general, (i) & (v) will be eukaryotic and (iii) & (iv) will be prokaryotic.

Preferred promoters are viral promoters e.g. from cytomegalovirus (CMV). The vector may also include transcriptional regulatory sequences (e.g. enhancers) in addition to the promoter and which interact functionally with the promoter. Preferred vectors include the immediate-early CMV enhancer/promoter, and more preferred vectors also include CMV intron A. The promoter is operably linked to a downstream sequence encoding an immunogen, such that expression of the immunogen-encoding sequence is under the promoter's control.

Where a marker is used, it preferably functions in a microbial host (e.g. in a prokaryote, in a bacteria, in a yeast). The marker is preferably a prokaryotic selectable marker (e.g. transcribed under the control of a prokaryotic promoter). For convenience, typical markers are antibiotic resistance genes.

The vector of the invention is preferably an autonomously replicating episomal or extrachromosomal vector, such as a plasmid.

The vector of the invention preferably comprises an origin of replication. It is preferred that the origin of replication is active in prokaryotes but not in eukaryotes.

Preferred vectors thus include a prokaryotic marker for selection of the vector, a prokaryotic origin of replication, but a eukaryotic promoter for driving transcription of the immunogen-encoding sequence. The vectors will therefore (a) be amplified and selected in prokaryotic hosts without polypeptide expression, but (b) be expressed in eukaryotic hosts without being amplified. This arrangement is ideal for nucleic acid immunization vectors.

The vector of the invention may comprise a eukaryotic transcriptional terminator sequence downstream of the coding sequence. This can enhance transcription levels. Where the coding sequence does not have its own, the vector of the invention preferably comprises a polyadenylation sequence. A preferred polyadenylation sequence is from bovine growth hormone.

The vector of the invention may comprise a multiple cloning site

In addition to sequences encoding the immunogen and a marker, the vector may comprise a second eukaryotic coding sequence. The vector may also comprise an IRES upstream of said second sequence in order to permit translation of a second eukaryotic polypeptide from the same transcript as the immunogen. Alternatively, the immunogen-coding sequence may be downstream of an IRES.

The vector of the invention may comprise unmethylated CpG motifs e.g. unmethylated DNA sequences which have in common a cytosine preceding a guanosine, flanked by two 5′ purines and two 3′ pyrimidines. In their unmethylated form these DNA motifs have been demonstrated to be potent stimulators of several types of immune cell.

Vectors may be delivered in a targeted way. Receptor-mediated DNA delivery techniques are described in, for example, references 118 to 123. Therapeutic compositions containing a nucleic acid are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g. for enhancing or inhibiting levels of the encoded gene product) and efficacy of transformation and expression are considerations which will affect the dosage required for ultimate efficacy. Where greater expression is desired over a larger area of tissue, larger amounts of vector or the same amounts re-administered in a successive protocol of administrations, or several administrations to different adjacent or close tissue portions may be required to effect a positive therapeutic outcome. In all cases, routine experimentation in clinical trials will determine specific ranges for optimal therapeutic effect.

Vectors can be delivered using gene delivery vehicles. The gene delivery vehicle can be of viral or non-viral origin (see generally references 124 to 127).

Viral-based vectors for delivery of a desired nucleic acid and expression in a desired cell are well known in the art. Exemplary viral-based vehicles include, but are not limited to, recombinant retroviruses (e.g. references 128 to 138), alphavirus-based vectors (e.g. Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532); hybrids or chimeras of these viruses may also be used), poxvirus vectors (e.g. vaccinia, fowlpox, canarypox, modified vaccinia Ankara, etc.), adenovirus vectors, and adeno-associated virus (AAV) vectors (e.g. see refs. 139 to 144). Administration of DNA linked to killed adenovirus [145] can also be employed.

Non-viral delivery vehicles and methods can also be employed, including, but not limited to, polycationic condensed DNA linked or unlinked to killed adenovirus alone [e.g. 145], ligand-linked DNA [146], eukaryotic cell delivery vehicles cells [e.g. refs. 147 to 151] and nucleic charge neutralization or fusion with cell membranes. Naked DNA can also be employed. Exemplary naked DNA introduction methods are described in refs. 152 and 153. Liposomes (e.g. immunoliposomes) that can act as gene delivery vehicles are described in refs. 154 to 158. Additional approaches are described in references 159 & 160.

Further non-viral delivery suitable for use includes mechanical delivery systems such as the approach described in ref. 160. Moreover, the coding sequence and the product of expression of such can be delivered through deposition of photopolymerized hydrogel materials or use of ionizing radiation [e.g. refs. 161 & 162]. Other conventional methods for gene delivery that can be used for delivery of the coding sequence include, for example, use of hand-held gene transfer particle gun [163] or use of ionizing radiation for activating transferred genes [161 & 164].

Delivery DNA using PLG {poly(lactide-co-glycolide)} microparticles is a particularly preferred method e.g. by adsorption to the microparticles, which are optionally treated to have a negatively-charged surface (e.g. treated with SDS) or a positively-charged surface (e.g. treated with a cationic detergent, such as CTAB).

### Antibodies

Antibodies against Y. pestis antigens can be used for passive immunisation [165]. Thus the invention provides an antibody that is specific for an antigen in the first, second, third or fourth antigen groups. The invention also provides the use of such antibodies in therapy. The invention also provides the use of such antibodies in the manufacture of a medicament. The invention also provides a method for treating a mammal comprising the step of administering an effective amount of a antibody of the invention. As described above for immunogenic compositions, these methods and uses allow a mammal to be protected against Y. pestis infection.

The term “antibody” includes intact immunoglobulin molecules, as well as fragments thereof which are capable of binding an antigen. These include hybrid (chimeric) antibody molecules [166, 167]; F(ab′)₂ and F(ab) fragments and Fv molecules; non-covalent heterodimers [168, 169]; single-chain Fv molecules (sFv) [170]; dimeric and trimeric antibody fragment constructs; minibodies [171, 172]; humanized antibody molecules [173-175]; and any functional fragments obtained from such molecules, as well as antibodies obtained through non-conventional processes such as phage display. Preferably, the antibodies are monoclonal antibodies. Methods of obtaining monoclonal antibodies are well known in the art.

Humanised or fully-human antibodies are preferred.

### General

Antigens are defined above by reference to “YPO” (or, in one case, “YPPCP”) nomenclature. This nomenclature refers to the numbering used in reference 11 for unique identification of open reading frames in the CO92 strain of Y. pestis. The basic reference sequence for any “YPO” or “YPPCP” number can easily be found in public gene databases. For instance, accession number NC_{—}003143 (GI:16120353) is the complete C092 genome sequence (4,653,728 bp), and the individual YPO sequences are given as “locus_tag” entries in the genome sequence's “features” section. Similarly, NC_{—}003132 (GI:16082679) is the complete sequence of the pPCP1 plasmid, and the “locus_tag” field gives the YPPCP number. Thus the nucleotide and amino acid sequences for any given YPO or YPPCP number can be established unambiguously. For convenience, however, the known sequences are included in a sequence listing filed herewith.

“GI” numbering is also used above. A GI number, or “GenInfo Identifier”, is a series of digits assigned consecutively to each sequence record processed by NCBI when sequences are added to its databases. The GI number bears no resemblance to the Accession number of the sequence record. When a sequence is updated (e.g. for correction, or to add more annotation or information) then it receives a new GI number. Thus the sequence associated with a given GI number is never changed.

Where the invention concerns an “epitope”, this epitope may be a B-cell epitope and/or a T-cell epitope. Such epitopes can be identified empirically (e.g. using PEPSCAN [176,177] or similar methods), or they can be predicted (e.g. using the Jameson-Wolf antigenic index [178], matrix-based approaches [179], TEPITOPE [180,181], neural networks [182], OptiMer & EpiMer [183, 184], ADEPT [185], Tsites [186], hydrophilicity [187], antigenic index [188] or the methods disclosed in reference 189, etc.). Epitopes are the parts of an antigen that are recognised by and bind to the antigen binding sites of antibodies or T-cell receptors, and they may also be referred to as “antigenic determinants”.

Variants of SEQ ID NOs include allelic variants, polymorphic forms, homologs, orthologs, paralogs, mutants, etc.

Polypeptides of the invention may, compared to the C092 reference sequence, include one or more (e.g. 1, 2, 3, 4, 5, 6, 7, 8, 9, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cystine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, etc.) single amino acid deletions relative to the C092 sequences. The polypeptides may also include one or more (e.g. 1, 2, 3, 4, 5, 6, 7, 8, 9, etc.) insertions (e.g. each of 1, 2, 3, 4 or 5 amino acids) relative to the C092 sequences.

Where an antigen “domain” is omitted, this may involve omission of a signal peptide, of a cytoplasmic domain, of a transmembrane domain, of an extracellular domain, etc.

The term “comprising” encompasses “including” as well as “consisting” e.g. a composition “comprising” X may consist exclusively of X or may include something additional e.g. X+Y.

The term “about” in relation to a numerical value x means, for example, x±10%.

If desired, antigens can be conjugated to a carrier protein in order to enhance immunogenicity.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref 190. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref 191.

### MODES FOR CARRYING OUT THE INVENTION

The methods used to select and obtain the antigens used in the invention are disclosed in reference 9.

### Mouse Immunisation Studies

23 antigens were chosen for further investigation. Combinations of 2-4 antigens were tested, although some antigens were tested individually. In total, nineteen groups of 10 mice (Swiss Webster, 5-7 week-old females) were immunized at two week intervals with 3 i.p. doses of antigen combination (containing 10 μg of each antigen), formulated with MF59+10 μg CpG (ODN 1826). Recent studies showed that antigen formulations with MF59 adjuvant are, in general, more immunogenic than alum formulations. A group of 10 adjuvant-only immunized mice and a group of mice immunized with the F1-V antigen fusion (an antigen known to be protective in mice) were also included as negative and positive controls respectively. Approximately 4 weeks after the third immunization dose, animals were challenged subcutaneously with 75 LD50s of the virulent Y. pestis CO92 strain and survival was monitored for 20 days post infection. The group immunized with F1-V showed 90% survival.

Table 1 shows animal survival and time to death for each immunized mouse group. Three animal groups (groups 6, 8 and 14), immunized with antigens found to be positive in the opsonophagocytosis and/or blood bactericidal assays (OPA/BBA), showed a 20% increase in the proportion of survivors. Two of them (groups 8 and 14) also showed an increase in their times-to-death as compared to the control group. Four further groups (2, 4, 11, 20), immunized with antigens found to be positive in the OPA/BBA assays, showed a 10% increase in the proportion of survivors. One group of animals (group 15) immunized with the combination of the three antigens identified by the proteomic approach showed a significant difference in the proportion of surviving animals (50% survival) and in the survival curve, as compared to the negative control group. This combination of antigens will therefore be useful alone and in combination with the F1-V vaccine.

It will be understood that the invention has been described by way of example only and modifications may be made whilst remaining within the scope and spirit of the invention.

TABLE 1
Protective activity of the 19 antigen combinations against subcutaneous challenge

| | | | | Survival Time | | | | |
|---|---|---|---|---|---|---|---|---|
| Immunized | Number of | | Time to death | (days) | | Pvalues¹ | | |
| group | Survivors/ | Survival | (days) | | Mean | Survival | Days to | Survival |
| Gene ID | Immunized | (%) | Mean (+/−SD) | Median | (+/−SEM) | Rate | death | Curves |
| Negative | 0/9 | 0 | 6.6 (+/−2.5) | 6 | 6.6 (+/−0.8) | | | |
| Control | | | | | | | | |
| 1 | 0/10 | 0 | 5.4 (+/−1.2 | 5.5 | 5.4 (+/−0.4) | 1.000 | 0.259 | 0.221 |
| YPO0086 | | | | | | | | |
| 2 | 1/10 | 10 | 5.8 (+/−1.4) | 6 | 7.3 (+/−1.6) | 0.526 | 0.459 | 0.911 |
| YPO0499 | | | | | | | | |
| 3 | 0/10 | 0 | 7.8 (+/−2.6) | 7 | 7.8 (+/−0.8) | 1.000 | 0.224 | 0.272 |
| YPO0505 | | | | | | | | |
| 4 | 1/10 | 10 | 6.4 (+/−2.2) | 6 | 7.9 (+/−1.6) | 0.526 | 0.916 | 0.432 |
| YPO0809 | | | | | | | | |
| 5 | 1/10 | 10 | 6.7 (+/−1.6) | 7 | 8.1 (+/−1.5) | 0.526 | 0.916 | 0.431 |
| YPO1070 | | | | | | | | |
| 6 | 2/10 | 20 | 6.3 (+/−2.5) | 6 | 9.2 (+/−2.1) | 0.263 | 0.777 | 0.288 |
| YPO1123 | | | | | | | | |
| 7 | 0/10 | 0 | 6.8 (+/−1.4) | 6.5 | 6.8 (+/−0.4) | 1.000 | 0.811 | 0.989 |
| YPO1604 | | | | | | | | |
| 8 | 2/10 | 20 | 7.9 (+/−3.0) | 9 | 10.5 (+/−2.0) | 0.263 | 0.223 | 0.053 |
| YPO2881 | | | | | | | | |
| 9 | 0/10 | 0 | 6.1 (+/−1.8) | 6 | 6.1 (+/−0.6) | 1.000 | 0.656 | 0.571 |
| YPO3061 | | | | | | | | |
| 10 | 0/10 | 0 | 8.2 (+/−4.4) | 6.5 | 8.2 (+/−1.4) | 1.000 | 0.109 | 0.272 |
| YPO3489 | | | | | | | | |
| 11 | 1/10 | 10 | 6.0 (+/−1.9) | 6 | 7.5 (+/−1.6) | 0.526 | 0.596 | 0.758 |
| YPO3631 | | | | | | | | |
| 12 | 0/10 | 0 | 6.7 (+/−3.1) | 6 | 6.7 (+/−1.0) | 1.000 | 0.888 | 0.918 |
| YPO4003 | | | | | | | | |
| 14 | 2/10 | 20 | 7.4 (+/−2.5) | 7 | 10.1 (+/−2.0) | 0.263 | 0.449 | 0.074 |
| YPO0499 | | | | | | | | |
| YPO1604 | | | | | | | | |
| YPO3489 | | | | | | | | |
| YPO4003 | | | | | | | | |
| 15 | 5/10 | 50 | 5.8 (+/−0.8) | — | 13.4 (+/−2.7) | 0.022 | 0.543 | 0.030 |
| YPO0505 | | | | | | | | |
| YPO0499 | | | | | | | | |
| YPO0502 | | | | | | | | |
| 16 | 0/10 | 0 | 6.8 (+/−1.1) | 7 | 6.8 (+/−0.4) | 1.000 | 0.811 | 0.917 |
| YPO1211 | | | | | | | | |
| YPO0015 | | | | | | | | |
| YPO0457 | | | | | | | | |
| 17 | 0/10 | 0 | 5.9 (+/−0.6) | 6 | 5.9 (+/−0.2) | 1.000 | 0.521 | 0.479 |
| YPO3674 | | | | | | | | |
| (domain) | | | | | | | | |
| YPO1951 | | | | | | | | |
| YPO1526 | | | | | | | | |
| YPO1686 | | | | | | | | |
| 18 | 0/10 | 0 | 6.2 (+/−1.2) | 6.5 | 6.2 (+/−0.4) | 1.000 | 0.728 | 0.654 |
| YPO0499 | | | | | | | | |
| YPO1604 | | | | | | | | |
| 19 | 0/10 | 0 | 7.3 (+/−2.4) | 7 | 7.3 (+/−0.7) | 1.000 | 0.467 | 0.489 |
| YPO3489 | | | | | | | | |
| YPO4003 | | | | | | | | |
| 20 | 1/10 | 10 | 5.4 (+/−1.9) | 5 | 7.0 (+/−1.7) | 0.526 | 0.290 | 0.881 |
| YPO1411 | | | | | | | | |
| YPO3935 | | | | | | | | |
| YPO3982 | | | | | | | | |

¹Pvalues were determined by pairwise comparison of each group with the negative control group. Significance of differences in Survival Rates, Survival curves and Mean Time to Death were determined by using Fisher Exact tests, Log-Rank tests and T-tests, respectively

### BRIEF DESCRIPTION OF SEQUENCE LISTING

| SEQ ID NO: | Name | GI |
|---|---|---|
| 1 | YPO0512 | 16120843 |
| 2 | YPO0086 | 16120437 |
| 3 | YPO0563 | 16120891 |
| 4 | YPO0809 | 16121121 |
| 5 | YPO0860 | 16121168 |
| 6 | YPO1070 | 16121371 |
| 7 | YPO1123 | 16121423 |
| 8 | YPO1411 | 16121691 |
| 9 | YPO1604 | 16121872 |
| 10 | YPO2881 | 16123073 |
| 11 | YPO3061 | 16123238 |
| 12 | YPO3065 | 16123242 |
| 13 | YPO3343 | 16123493 |
| 14 | YPO3361 | 16123511 |
| 15 | YPO3382 | 16123531 |
| 16 | YPO3430 | 16123579 |
| 17 | YPO3489 | 16123635 |
| 18 | YPO3559 | 16123703 |
| 19 | YPO3631 | 16123773 |
| 20 | YPO3935 | 16124063 |
| 21 | YPO4003 | 16124128 |
| 22 | YPO3644 | 16123786 |
| 23 | YPO3643 | 16123785 |
| 24 | YPO3536 | 16123682 |
| 25 | YPO3050 | 16123227 |
| 26 | YPO2674 | 16122879 |
| 27 | YPO2615 | 16122828 |
| 28 | YPO2342 | 16122566 |
| 29 | YPO2292 | 16122516 |
| 30 | YPO1746 | 16122003 |
| 31 | YPO1507 | 16121780 |
| 32 | YPO1435 | 16121713 |
| 33 | YPO1053 | 16121353 |
| 34 | YPO0819 | 16121130 |
| 35 | YPO0570 | 16120899 |
| 36 | YPO0502 | 16120832 |
| 37 | YPO0501 | 16120831 |
| 38 | YPO0468 | 16120797 |
| 39 | YPO0351 | 16120686 |
| 40 | YPO0233 | 16120571 |
| 41 | YPO0216 | 16120553 |
| 42 | YPO0203 | 16120542 |
| 43 | YPO0195 | 16120534 |
| 44 | YPO1002 | 16120449 |
| 45 | YPO0067 | 16120418 |
| 46 | YPO0015 | 16120369 |
| 47 | YPO4111 | 16124219 |
| 48 | YPMT1.84 | 16082876 |
| 49 | YPCD1.31c | 5832451 |
| 50 | YPPCP1.07 | 16082686 |
| 51 | YPO1052 | 16121352 |
| 52 | YPO4070 | 16124183 |
| 53 | YPO0494 | 16120824 |
| 54 | YPO0663 | 16120988 |
| 55 | YPO1222 | 16121511 |
| 56 | YPO1906 | 16122154 |
| 57 | YPO2905 | 16123096 |
| 58 | YPO3375 | 16123524 |
| 59 | YPMT1.42 | 16082828 |
| 60 | Linker | — |
| 61 | YPO0457 | 16120786 |
| 62 | YPO0514 | 16120845 |
| 63 | YPO0694 | 16121015 |
| 64 | YPO0805 | 16121117 |
| 65 | YPO0982 | 16121286 |
| 66 | YPO1354 | 16121634 |
| 67 | YPO1408 | 16121688 |
| 68 | YPO1792 | 16122046 |
| 69 | YPO2506 | 16122727 |
| 70 | YPO2713 | 16122917 |
| 71 | YPO2950 | 16123133 |
| 72 | YPO3026 | 16123203 |
| 73 | YPO3417 | 16123566 |
| 74 | YPO3551 | 16123695 |
| 75 | YPO3646 | 16123788 |
| 76 | YPO3982 | 16124109 |
| 77 | YPO0065 | 16120416 |
| 78 | YPO0499 | 16120829 |
| 79 | YPO0505 | 16120835 |
| 80 | YPO0500 | 16120830 |
| 81 | YPO0503 | 16120833 |
| 82 | YPO0506 | 16120836 |
| 83 | YPO0508 | 16120838 |
| 84 | YPO0509 | 16120839 |
| 85 | YPO3579 | 16123723 |
| 86 | YPO4040 | 16124160 |
| 87 | YPO0496 | 16120826 |
| 88 | YPO1224 | 16121513 |
| 89 | YPO3553 | 16123697 |
| 90 | YPO3987 | 16124114 |
| 91 | YPO2190 | 16122420 |

### REFERENCES

[1] Perry & Fetherstone 1997 Clin Microbiol Rev 10:35-66.
[2] Inglesby et al. 2001 Jama 283:2281-90.
[3] Titball & Williamson 2001 Vaccine 19:4175-84.
[4] Drozdov et al. 1995 J Med Microbiol 42:264-268.
[5] Worsham et al. 1995 Contrib Microbiol Immunol 13:325-328.
[6] Winter 1960 Bull W.H.O. 23:408-409.
[7] Miller et al. 1998 FEMS Immunol Med Microbiol 21:213-221.
[8] Anderson et al. 1996 Infect Immunol 64:4580-4585.
[9] WO2007/049155
[10] Tanabe et al. (2006) Infect. Immun. 74(6):3687-3691.
[11] Parkhill et al. 2001 Nature 413:523-7.
[12] Smither et al. (2006) J. Microbiol Methods E-publication 17/07/06.
[13] Galyov et al. (1990) FEBS Lett 277(1-2):230-2.
[14] Adair et al. 2000 J Clin Microbiol 38:1516-1519.
[15] Honko et al. (2006) Infect. Immun 74(2):1113-1120.
[16] Segal et al. 2005 FEMS Microbiol Rev 29(1):65-81.
[17] Das et al. 2003 In Silico Biol. 3:287-300.
[18] Brand et al. 1994 Mol Microbiol 14(4):797-808.
[19] VanRheenen et al. 2004 Infect Immun 72(10):5972-82.
[20] Sexton et al. 2004 Infect Immun 72(10):5983-92.
[21] Zheng et al. 2005 Infect Immun 73(7):4127-37.
[22] Powell et al. (2005) Biotechnol Prog 21(5):1490-510.
[23] Drancourt et al. (2004) Emerg Infect Dis 10:1585-92.
[24] Deng et al. 2002 J Bacteria 184(16):4601-11.
[25] Song et al. (2004) DNA Res. 11:179-97.
[26] Chain et al. (2004) PNAS USA 101(38):13826-13831.
[27] Vaccine Design . . . (1995) eds. Powell & Newman. ISBN: 030644867X. Plenum.
[28] WO00/23105.
[29] WO90/14837.
[30] U.S. Pat. No. 5,057,540.
[31] WO96/33739.
[32] EP-A-0109942.
[33] WO96/11711.
[34] WO00/07621.
[35] Barr et al. (1998) Advanced Drug Delivery Reviews 32:247-271.
[36] Sjolanderet et al. (1998) Advanced Drug Delivery Reviews 32:321-338.
[37] Niikura et al. (2002) Virology 293:273-280.
[38] Lenz et al. (2001) J Immunol 166:5346-5355.
[39] Pinto et al. (2003) J Infect Dis 188:327-338.
[40] Gerber et al. (2001) J Virol 75:4752-4760.
[41] WO03/024480
[42] WO03/024481
[43] Gluck et al. (2002) Vaccine 20:B10-B16.
[44] EP-A-0689454.
[45] Johnson et al. (1999) Bioorg Med Chem Lett 9:2273-2278.
[46] Evans et al. (2003) Expert Rev Vaccines 2:219-229.
[47] Meraldi et al. (2003) Vaccine 21:2485-2491.
[48] Pajak et al. (2003) Vaccine 21:836-842.
[49] Kandimalla et al. (2003) Nucleic Acids Research 31:2393-2400.
[50] WO02/26757.
[51] WO99/62923.
[52] Krieg (2003) Nature Medicine 9:831-835.
[53] McCluskie et al. (2002) FEMS Immunology and Medical Microbiology 32:179-185.
[54] WO98/40100.
[55] U.S. Pat. No. 6,207,646.
[56] U.S. Pat. No. 6,239,116.
[57] U.S. Pat. No. 6,429,199.
[58] Kandimalla et al. (2003) Biochemical Society Transactions 31 (part 3):654-658.
[59] Blackwell et al. (2003) J Immunol 170:4061-4068.
[60] Krieg (2002) Trends Immunol 23:64-65.
[61] WO01/95935.
[62] Kandimalla et al. (2003) BBRC 306:948-953.
[63] Bhagat et al. (2003) BBRC 300:853-861.
[64] WO03/035836.
[65] WO95/17211.
[66] WO98/42375.
[67] Beignon et al. (2002) Infect Immun 70:3012-3019.
[68] Pizza et al. (2001) Vaccine 19:2534-2541.
[69] Pizza et al. (2000) Int J Med Microbiol 290:455-461.
[70] Scharton-Kersten et al. (2000) Infect Immun 68:5306-5313.
[71] Ryan et al. (1999) Infect Immun 67:6270-6280.
[72] Partidos et al. (1999) Immunol Lett 67:209-216.
[73] Peppoloni et al. (2003) Expert Rev Vaccines 2:285-293.
[74] Pine et al. (2002) J Control Release 85:263-270.
[75] Domenighini et al. (1995) Mol Microbiol 15:1165-1167.
[76] WO99/40936.
[77] WO99/44636.
[78] Singh et al] (2001) J Cont Release 70:267-276.
[79] WO99/27960.
[80] U.S. Pat. No. 6,090,406
[81] U.S. Pat. No. 5,916,588
[82] EP-A-0626169.
[83] WO99/52549.
[84] WO01/21207.
[85] WO01/21152.
[86] Andrianov et al. (1998) Biomaterials 19:109-115.
[87] Payne et al. (1998) Adv Drug Delivery Review 31:185-196.
[88] Stanley (2002) Clin Exp Dermatol 27:571-577.
[89] Jones (2003) Curr Opin Investig Drugs 4:214-218.
[90] WO99/11241.
[91] WO94/00153.
[92] WO98/57659.
[93] European patent applications 0835318, 0735898 and 0761231.
[94] Huang et al. (2002) J Clin Microbiol. 40(4):1164-73.
[95] Grif et al. (2003) Diagn Microbiol Infect Dis 47(1):313-20.
[96] Zhou et al. (2004) Bacteriol 186(15):5147-52.
[97] WO98/24912
[98] WO99/27961
[99] WO02/074244
WO02/064162
WO03/028760
Gennaro (2000) Remington: The Science and Practice of Pharmacy. 20th edition, ISBN: 0683306472.
Williamson et al. (2005) Infect Immun 73(9):5978-87.
Donnelly et al. (1997) Annu Rev Immunol 15:617-648.
Strugnell et al. (1997) Immunol Cell Biol 75(4):364-369.
Cui (2005) Adv Genet. 54:257-89.
Robinson & Torres (1997) Seminars in Immunol 9:271-283.
Brunham et al. (2000) J Infect Dis 181 Suppl 3:S538-43.
Svanholm et al. (2000) Scand J Immunol 51(4):345-53.
DNA Vaccination—Genetic Vaccination (1998) eds. Koprowski et al. (ISBN 3540633928).
Gene Vaccination: Theory and Practice (1998) ed. Raz (ISBN 3540644288).
Wang et al. (2004) Vaccine 22:3348-57.
Titball & Williamson (2004) Expert Opin Biol Ther 4:965-73.
Garmory et al. (2004) Vaccine 22:947-57.
Grosfeld et al. (2003) Infect Immun 71(1):374-83.
Williamson et al. (2002) Vaccine 20:2933-41.
Bennett et al. (1999) Vaccine 18(7-8):588-96.
Findeis et al., Trends Biotechnol. (1993) 11:202
Chiou et al. (1994) Gene Therapeutics: Methods And Applications Of Direct Gene Transfer. ed. Wolff
Wu et al., J. Biol. Chem. (1988) 263:621
Wu et al., J. Biol. Chem. (1994) 269:542
Zenke et al., Proc. Natl. Acad. Sci. (USA) (1990) 87:3655
Wu et al., J. Biol. Chem. (1991) 266:338
Jolly, Cancer Gene Therapy (1994) 1:51
Kimura, Human Gene Therapy (1994) 5:845
Connelly, Human Gene Therapy (1995) 1:185
Kaplitt, Nature Genetics (1994) 6:148
WO 90/07936
WO 94/03622
WO 93/25698
WO 93/25234
U.S. Pat. No. 5,219,740
WO 93/11230
WO 93/10218
U.S. Pat. No. 4,777,127
GB Patent No. 2,200,651
EP-A-0 345 242
WO 91/02805
WO 94/12649
WO 93/03769
WO 93/19191
WO 94/28938
WO 95/11984
WO 95/00655
Curiel, Hum. Gene Ther. (1992) 3:147
Wu, J. Biol. Chem. (1989) 264:16985
U.S. Pat. No. 5,814,482
WO 95/07994
WO 96/17072
WO 95/30763
WO 97/42338
WO 90/11092
U.S. Pat. No. 5,580,859
U.S. Pat. No. 5,422,120
WO 95/13796
WO 94/23697
WO 91/14445
EP 0524968
Philip, Mol. Cell. Biol. (1994) 14:2411
Woffendin, Proc. Natl. Acad. Sci. (1994) 91:11581
U.S. Pat. No. 5,206,152
WO 92/11033
U.S. Pat. No. 5,149,655
WO 92/11033
WO2005/047309.
Winter et al., (1991) Nature 349:293-99
U.S. Pat. No. 4,816,567
Inbar et al., (1972) Proc. Natl. Acad. Sci. U.S.A. 69:2659-62.
Ehrlich et al., (1980) Biochem 19:4091-96.
Huston et al., (1988) Proc. Natl. Acad. Sci. U.S.A. 85:5897-83.
Pack et al., (1992) Biochem 31, 1579-84.
Cumber et al., (1992) J. Immunology 149B, 120-26.
Riechmann et al., (1988) Nature 332, 323-27.
Verhoeyan et al., (1988) Science 239, 1534-36.
GB 2,276,169
Geysen et al. (1984) PNAS USA 81:3998-4002.
Carter (1994) Methods Mol Biol 36:207-23.
Jameson, B A et al. 1988, CABIOS 4(1):181-186.
Raddrizzani & Hammer (2000) Brief Bioinform 1(2):179-89.
De Lalla et al. (1999) J. Immunol. 163:1725-29.
Kwok et al. (2001) Trends Immunol 22:583-88.
Brusic et al. (1998) Bioinformatics 14(2):121-30
Meister et al. (1995) Vaccine 13(6):581-91.
Roberts et al. (1996) AIDS Res Hum Retroviruses 12(7):593-610.
Maksyutov & Zagrebelnaya (1993) Comput Appl Biosci 9(3):291-7.
Feller & de la Cruz (1991) Nature 349(6311):720-1.
Hopp (1993) Peptide Research 6:183-190.
Welling et al. (1985) FEBS Lett. 188:215-218.
Davenport et al. (1995) Immunogenetics 42:392-297.
Current Protocols in Molecular Biology (F. M. Ausubel et al., eds., 1987) Supplement 30
Smith & Waterman (1981) Adv. Appl. Math. 2: 482-489.

1. An immunogenic composition comprising two or more of isolated Y. pestis (YP) antigens, wherein the isolated YP antigens are selected from the group consisting of a YPO0499 antigen, a YPO0502 antigen, a YPO0505 antigen, a YPO0809 antigen, a YPO1070 antigen, a YPO1123 antigen, a YPO1604 antigen, a YPO2881 antigen, a YPO3489 antigen, a YPO1411 antigen, a YPO3935 antigen, a YPO3982 antigen and a YPO4003 antigen.

2. The immunogenic composition of claim 1, wherein the composition comprises an isolated YPO4003 antigen, an isolated YPO1604 antigen, an isolated YPO3489 antigen and an isolated YPO0499 antigen.

3. The immunogenic composition of claim 1, wherein the composition comprises an isolated YPO0499 antigen, an isolated YPO0502 antigen and an isolated YPO0505 antigen.

4. The immunogenic composition of claim 1, wherein the composition comprises an isolated YPO1070 antigen, an isolated YPO1123 antigen, an isolated YPO2881 antigen and an isolated YPO0809 antigen.

5. An immunogenic composition comprising a combination of Y. pestis antigens according to claim 1, said combination comprising a YPO1411 antigen, a YPO3935 antigen and a YPO3982 antigen.

6. An immunogenic composition comprising a combination of Y. pestis antigens, said combination comprising two or more antigens selected from the group consisting of: a YPO0065 antigen, a YPO0086 antigen, a YPO0496 antigen, a YPO0499 antigen, a YPO0501 antigen, a YPO0502 antigen, a YPO0505 antigen, a YPO0809 antigen, a YPO0860 antigen, a YPO1070 antigen, a YPO1123 antigen, a YPO1224 antigen, a YPO1411 antigen, a YPO1604 antigen, a YPO2506 antigen, a YPO2881 antigen, a YPO3935 antigen, a YPO3061 antigen, a YPO3065 antigen, a YPO3382 antigen, a YPO3489 antigen, a YPO3551 antigen, a YPO3553 antigen, a YPO3579 antigen, a YPO3631 antigen, a YPO3982 antigen, a YPO4003 antigen, a YPO3987 antigen, a YPO1354 antigen, a YPO2190 antigen and a YPO3417 antigen.

7. An immunogenic composition comprising a combination of Y. pestis antigens, said combination comprising two or more antigens selected from the group consisting of: (1) a YPO0512 antigen; (2) a YPO0563 antigen; and (3) a YPO3489 antigen.

8. An immunogenic composition comprising a combination of Y. pestis antigens, said combination comprising two or more antigens selected from the group consisting of (1) a YPO0512 antigen; (2) a YPO0563 antigen; (3) a YPO3489 antigen; (4) a YPO4003 antigen; and (5) a YPO1604 antigen.

9. An immunogenic composition comprising a combination of Y. pestis antigens, said combination comprising one or more antigens from the group consisting of: (1) a YPO0512 antigen; (2) a YPO0563 antigen; (3) a YPO3489 antigen; (4) a YPO4003 antigen; (5) a YPO1604 antigen; (6) a YPO3061 antigen; (7) a YPO3559 antigen; (8) a YPO3382 antigen; (9) a YPO0860 antigen; (10) a YPO0086 antigen; (11) a YPO3631 antigen; (12) a YPO2881 antigen; (13) a YPO3343 antigen; (14) a YPO3361 antigen; (15) a YPO3430 antigen; (16) a YPO1411 antigen; (17) a YPO3935 antigen; (18) a YPO0809 antigen; (19) a YPO1123 antigen; (20) a YPO3065 antigen; and (21) a YPO 1070 antigen.

10. An immunogenic composition comprising a combination of Y. pestis antigens, said combination comprising one or more antigens from the group consisting of: (1) a YPO0102 antigen; (2) a YPO0570 antigen; (3) a YPO1053 antigen; (4) a YPO1435 antigen; (5) a YPO2674 antigen; (6) a YPO2292 antigen; (7) a YPO3050 antigen; (8) a YPO2615 antigen; (9) a YPO1507 antigen; (10) a YPO4111 antigen; (11) a YPO0015 antigen; (12) a YPO0195 antigen; (13) a YPO2342 antigen; (14) a YPO0501 antigen; (15) a YPO0502 antigen; (16) a YPO0819 antigen; (17) a YPO3644 antigen; (18) a YPO1746 antigen; (19) a YPO0351 antigen; (20) a YPO0468 antigen; (21) a YPO0203 antigen; (22) a YPO0216 antigen; (23) a YPO3536 antigen; (24) a YPO0233 antigen; (25) a YPO0067 antigen; (26) a YPO3643 antigen; (27) a YPO3375 antigen; (28) a YPO0494 antigen; (29) a YPO 1052 antigen; (30) a YPO 1906 antigen; (31) a YPO0663 antigen; (32) a YPO1222 antigen; (33) a YPO2905 antigen; (34) a YPO4070 antigen; (35) a YPPCP1.07 antigen; and/or (36) a YPMT1.42 antigen.

11. An immunogenic composition comprising a combination of Y. pestis antigens, said combination comprising one or more antigens from the group consisting of: (1) a YPO0457 antigen; (2) a YPO0514 antigen; (3) a YPO0694 antigen; (4) a YPO0805 antigen; (5) a YPO0982 antigen; (6) a YPO 1354 antigen; (7) a YPO 1408 antigen; (8) a YPO 1792 antigen; (9) a YPO2506 antigen; (10) a YPO2713 antigen; (11) a YPO2950 antigen; (12) a YPO3026 antigen; (13) a YPO3417 antigen; (14) a YPO3551 antigen; (15) a YPO3646 antigen; (16) a YPO3982 antigen; (17) a YPO0065 antigen; (18) a YPO0499 antigen; (19) a YPO0505 antigen; (20) a YPO0500 antigen; (21) a YPO0503 antigen; (22) a YPO0506 antigen; (23) a YPO0508 antigen; (24) a YPO0509 antigen; (25) a YPO3579 antigen and/or (26) a YPO4040 antigen.

12. An immunogenic composition comprising a combination of Y. pestis antigens, said combination comprising one or more antigens from the group consisting of: (1) a YPO0496 antigen; (2) a YPO1224 antigen; (3) a YPO3553 antigen; (4) a YPO3987 antigen; and (5) a YPO2190 antigen.

13. An immunogenic composition comprising a combination of Y. pestis antigens, said combination including one or more antigens selected from the group of claim 9.

14. An immunogenic composition comprising a combination of Y. pestis antigens, said combination including one or more antigens selected from the group of claim 9 and one or both of Y. pestis F1 and V antigens.

15. An immunogenic composition comprising a combination of Y. pestis antigens, said combination including one or more antigens selected from the group of claim 10 and one or both of Y. pestis F1 and V antigens.

16. An immunogenic composition comprising a combination of Y. pestis antigens, said combination including one or more antigens selected from the group of claim 9, and one or both of Y. pestis F1 and V antigens.

17. An immunogenic composition comprising a combination of Y. pestis antigens, said combination including one or more antigens selected from the group one or more antigens of the group of claim 11.

18. An immunogenic composition comprising a combination of Y. pestis antigens, said combination including one or more antigens selected from the group of claim 10.

19. An immunogenic composition comprising a combination of Y. pestis antigens, said combination including one or more antigens selected from the group of claim 11 and one or both of Y. pestis F1 and V antigens.

20. An immunogenic composition comprising a combination of Y. pestis antigens, said combination including one or more antigens selected from the group of claim 11, and one or both of Y. pestis F1 and V antigens.

21. An immunogenic composition comprising a combination of Y. pestis antigens, said combination including one or more antigens of the group of claim 12.

22. An immunogenic composition comprising a combination of Y. pestis antigens, said combination including one or more antigens selected from the group of claim 10 and further comprising one or more antigens selected from the group consisting of (1) a YPO0496 antigen; (2) a YPO1224 antigen; (3) a YPO3553 antigen; (4) a YPO3987 antigen; and (5) a YPO2190 antigen.

23. An immunogenic composition comprising a combination of Y. pestis antigens, said combination including one or more antigens selected from the group of claim 11 and further comprising one or more antigens selected from the group consisting of (1) a YPO0496 antigen; (2) a YPO1224 antigen; (3) a YPO3553 antigen; (4) a YPO3987 antigen; and (5) a YPO2190 antigen.

24. An immunogenic composition comprising a combination of Y. pestis antigens, said combination including one or more antigens selected from the group of claim 12 and one or both of Y. pestis F1 and V antigens.

25. An immunogenic composition comprising a combination of Y. pestis antigens, said combination including one or more antigens selected from the group of claim 9, one or more antigens selected from the group consisting of (1) a YPO0496 antigen; (2) a YPO1224 antigen; (3) a YPO3553 antigen; (4) a YPO3987 antigen; and (5) a YPO2190 antigen and one or both of Y. pestis F1 and V antigens.

26. An immunogenic composition comprising a combination of Y. pestis antigens, said combination including one or more antigens selected from the group of claim 6, and one or both of Y. pestis F1 and V antigens.

27. An immunogenic composition comprising a combination of Y. pestis antigens according to claim 1, further comprising one or both of Y. pestis F1 and V antigens.

28. (canceled)

29. The immunogenic composition of claim 1, wherein at least one of the antigens is a fusion protein.

30. The immunogenic composition of claim 1, wherein at least two of the antigens are expressed as a single polypeptide chain.

31. The immunogenic composition of claim 1, wherein the composition includes one or more immunoregulatory agents.

32. A method of raising an immune response in a mammal, the method comprising administering a composition according to claim 1 to the mammal.

33. (canceled)

34. The method of claim 32, wherein the composition comprises the following isolated YP antigens: a YPO4003 antigen, a YPO 1604 antigen, a YPO3489 antigen and a YPO0499 antigen.

35. (canceled)

36. The method of claim 32, wherein the composition comprises the following isolated YP antigens: a YPO0499 antigen, a YPO0502 antigen and a YPO0505 antigen.

37. (canceled)

38. The method of claim 32, wherein the composition comprises the following isolated YP antigens: a YPO1070 antigen, a YPO1123 antigen, a YPO2881 antigen and a YPO0809 antigen.

39-49. (canceled)

50. A method of raising an immune response in a mammal, the method comprising administering a composition comprising an isolated YP antigen selected from the group consisting of a YPO0499 antigen, a YPO0502 antigen, and combinations thereof.

51-52. (canceled)

53. An antibody that is specific for an antigen according to claim 1.
